(19) United States
(12) Patent Application Publication
(10) Pub. No.: US 2019/0015305 A1
(43) Pub. Date: Jan. 17, 2019

(54) SYSTEMS AND METHODS FOR WHITENING TEETH

(71) Applicant: Evolve Dental Technologies, Inc., Irvine, CA (US)

(72) Inventor: Rodger Kurthy, Coto de Caza, CA (US)

(21) Appl. No.: 16/005,518

(22) Filed: Jun. 11, 2018

Related U.S. Application Data
(60) Provisional application No. 62/517,969, filed on Jun. 11, 2017.

(51) Int. Cl. A61K 8/22; A61K 8/42; A61Q 11/00

(52) U.S. Cl. CPC A61K 8/22; A61K 8/42; A61K 2800/884; A61K 2800/87; A61Q 11/00

(57) ABSTRACT

Systems and methods for whitening teeth. The method may include making trays for a patient and providing the patient with instructions and products for at home whitening. The method may include performing at home nighttime whitening using carbamide peroxide, or any other whitening product that breaks down slowly. The method may include performing at home daytime whitening using hydrogen peroxide, or any other whitening product that breaks down quickly. The daytime whitening may use a chemically accelerated hydrogen peroxide or a mixture of hydrogen peroxide and carbamide peroxide. The at home daytime whitening may be performed at least partially concurrently with the at home nighttime whitening. The at home daytime whitening may be performed after the at home nighttime whitening.

### INCORPORATION BY REFERENCE TO ANY PRIORITY APPLICATIONS

This application claims priority benefit of U.S. Provisional Patent Application No. 62/517969, filed on Jun. 11, 2017 (Dkt. No. EVOLV.009PR), which is incorporated herein by reference in its entirety for all purposes. The entire contents of all of the above applications are incorporated by reference herein and made a part of this specification. Any and all applications for which foreign or domestic priority claim is identified in the Application Data Sheet as filed with the present application are hereby incorporated.

### BACKGROUND

### Field of the Invention

The present application relates generally to systems and methods for whitening teeth, and more specifically, relates to systems and methods for whitening teeth using hydrogen peroxide and carbamide peroxide.

### Description of the Related Art

Tooth whitening is a cosmetic, non-therapeutic, service offered by some dental professionals. Different methods or combinations thereof may be used to cosmetically whiten the teeth, including, for example, chemical whitening, and/or laser whitening.

### SUMMARY

In some embodiments, a method of cosmetically whitening teeth of a patient comprises whitening the teeth at night using a carbamide peroxide-based whitening product, the carbamide peroxide-based whitening product having a concentration of carbamide peroxide of about 16% or less, and after at least one night of whitening the teeth at night using the carbamide peroxide-based whitening product, whitening the teeth during the day using a hydrogen peroxide-based whitening product, the hydrogen peroxide-based whitening product having a concentration of about 6% or less. Whitening the teeth at night comprises: placing a quantity of the carbamide peroxide-based whitening product in a tray customized to the teeth of the patient; placing the tray containing the quantity of the carbamide peroxide-based whitening product over the teeth of the patient before the patient sleeps; and leaving the tray containing the quantity of the carbamide peroxide-based whitening product over the teeth of the patient for a length of time while the patient sleeps. Whitening the teeth during the day comprises: placing a quantity of the hydrogen peroxide-based whitening product in the tray customized to the teeth of the patient; placing the tray containing the quantity of the hydrogen peroxide-based whitening product over the teeth of the patient while the patient is awake during the day; and leaving the tray containing the quantity of the hydrogen peroxide-based whitening product over the teeth of the patient for a length of time while the patient is awake during the day.

Whitening the teeth at night using the carbamide peroxide-based whitening product may be performed each night for two weeks. Whitening the teeth during the day using the hydrogen peroxide-based whitening product may be performed after the step of whitening the teeth at night using the carbamide peroxide-based whitening product has been performed each night for two weeks. Whitening the teeth during the day using the hydrogen peroxide-based whitening product may be performed each day for one week. Whitening the teeth during the day using the hydrogen peroxide-based whitening product may be performed once a day for at least 60 minutes. Whitening the teeth during the day using the hydrogen peroxide-based whitening product may be performed at least twice a day for at least 60 minutes. Whitening the teeth at night using the carbamide peroxide-based whitening product may be performed each night for between one and three weeks. Whitening the teeth during the day using the hydrogen peroxide-based whitening product may be performed after the step of whitening the teeth at night using the carbamide peroxide-based whitening product has been performed each night for between one and three weeks. Whitening the teeth during the day using the hydrogen peroxide-based whitening product may be performed each day for at least one week. Whitening the teeth during the day using the hydrogen peroxide-based whitening product may be performed once a day for between about 15-60 minutes. Whitening the teeth during the day using the hydrogen peroxide-based whitening product may be performed at least twice a day for between about 15-60 minutes. Whitening the teeth during the day using the hydrogen peroxide-based whitening product may be performed at least three times a day for between about 15-60 minutes. Whitening the teeth during the day using the hydrogen peroxide-based whitening product may be started after the step of whitening the teeth at night using the carbamide peroxide-based whitening product has been performed each night for about one week. Whitening the teeth during the day using the hydrogen peroxide-based whitening product may be started after the step of whitening the teeth at night using the carbamide peroxide-based whitening product has been performed each night for about two weeks. Whitening the teeth during the day using the hydrogen peroxide-based whitening product may be performed each day for at least one week. Whitening the teeth during the day using the hydrogen peroxide-based whitening product is performed once a day for between about 15-60 minutes. Whitening the teeth during the day using the hydrogen peroxide-based whitening product may be performed at least twice a day for between about 15-60 minutes. Whitening the teeth during the day using the hydrogen peroxide-based whitening product may be performed at least three times a day for between about 15-60 minutes. Whitening the teeth during the day using the hydrogen peroxide-based whitening product may be performed each day for at least one week. Whitening the teeth during the day using the hydrogen peroxide-based whitening product may be performed once a day for between about 15-60 minutes. Whitening the teeth during the day using the hydrogen peroxide-based whitening product may be performed at least twice a day for between about 15-60 minutes. Whitening the teeth during the day using the hydrogen peroxide-based whitening product may be performed at least three times a day for between about 15-60 minutes. The carbamide peroxide-based whitening product may comprise a chemical accelerant. The hydrogen peroxide-based whitening product may comprise a chemical accelerant.

In some embodiments, a method of cosmetically whitening teeth of a patient, the method comprising: whitening the teeth at night using a nighttime whitening product, and after at least one night of whitening the teeth at night using the nighttime whitening product. Whitening the teeth at night comprises: placing a quantity of the nighttime whitening product in a tray customized to the teeth of the patient; placing the tray containing the quantity of the nighttime whitening product over the teeth of the patient before the patient sleeps; and leaving the tray containing the quantity of the nighttime whitening product over the teeth of the patient for a length of time while the patient sleeps. Whitening the teeth during the day using a daytime whitening product, wherein whitening the teeth during the day comprises: placing a quantity of the daytime whitening product in the tray customized to the teeth of the patient; placing the tray containing the quantity of the daytime whitening product over the teeth of the patient while the patient is awake during the day; and leaving the tray containing the quantity of the daytime whitening product over the teeth of the patient for a length of time while the patient is awake during the day.

The nighttime whitening product may comprise a carbamide peroxide-based whitening product having a concentration of carbamide peroxide of about 35%. The daytime whitening product may comprise a hydrogen peroxide-based whitening product having a concentration of hydrogen peroxide of less than about 25%. The daytime whitening product may comprise a hydrogen peroxide-based whitening product having a concentration of hydrogen peroxide of about 16% and a carbamide peroxide-based whitening product having a concentration of carbamide peroxide of about 35%. The daytime whitening product may comprise about 75% of the hydrogen peroxide-based whitening product and about 25% of the carbamide peroxide-based whitening product. Whitening the teeth at night using the nighttime whitening product may be performed each night for two weeks. Whitening the teeth during the day using the daytime whitening product may be performed after the step of whitening the teeth at night using the nighttime whitening product has been performed each night for two weeks. Whitening the teeth during the day using the daytime whitening product may be performed each day for one week. Whitening the teeth during the day using the daytime whitening product may be performed once a day for at least 60 minutes. Whitening the teeth during the day using the daytime whitening product may be performed at least twice a day for at least 60 minutes. Whitening the teeth at night using the nighttime whitening product may be performed each night for between one and three weeks. Whitening the teeth during the day using the daytime whitening product may be performed after the step of whitening the teeth at night using the nighttime whitening product has been performed each night for between one and three weeks. Whitening the teeth during the day using the daytime whitening product may be performed each day for at least one week. Whitening the teeth during the day using the daytime whitening product may be performed once a day for between about 15-60 minutes. Whitening the teeth during the day using the daytime whitening product may be performed at least twice a day for between about 15-60 minutes. Whitening the teeth during the day using the daytime whitening product may be performed at least three times a day for between about 15-60 minutes. Whitening the teeth during the day using the daytime whitening product may be started after the step of whitening the teeth at night using the nighttime whitening product has been performed each night for about one week. Whitening the teeth during the day using the daytime whitening product may be started after the step of whitening the teeth at night using the nighttime whitening product has been performed each night for about two weeks. Whitening the teeth during the day using the daytime whitening product may be performed each day for at least one week. Whitening the teeth during the day using the daytime whitening product may be performed once a day for between about 15-60 minutes. Whitening the teeth during the day using the daytime whitening product may be performed at least twice a day for between about 15-60 minutes. Whitening the teeth during the day using the daytime whitening product may be performed at least three times a day for between about 15-60 minutes. Whitening the teeth during the day using the daytime whitening product may be performed each day for at least one week. Whitening the teeth during the day using the daytime whitening product may be performed once a day for between about 15-60 minutes. Whitening the teeth during the day using the daytime whitening product may be performed at least twice a day for between about 15-60 minutes. Whitening the teeth during the day using the daytime whitening product may be performed at least three times a day for between about 15-60 minutes. The nighttime whitening product may comprise a chemical accelerant. The daytime whitening product may comprise a chemical accelerant.

### BRIEF DESCRIPTION OF THE DRAWINGS

FIG. 1 shows a flow chart of one embodiment of a method for daytime vital tray whitening of teeth.

FIG. 2 shows a flow chart of one embodiment of a method for nighttime vital tray whitening of teeth.

FIG. 3 shows a flow chart of one embodiment of a method for in-office power whitening of teeth.

FIG. 4 shows a flow chart of one embodiment of a method for combination whitening of teeth.

FIG. 5 shows a flow chart of one embodiment of a method for Deep Bleaching whitening of teeth.

FIG. 6 shows a flow chart of one embodiment of a method for low concentration whitening of teeth.

FIG. 7 shows a flow chart of one embodiment of a method for alternative combination whitening of teeth.

### DETAILED DESCRIPTION

### How Teeth Become Dark

Teeth may acquire color (e.g., darkness) due to molecules, e.g., dark molecules, collecting or accumulating within tooth structure (e.g., adhering, bonding, etc. to the tooth structure, including the internal tooth structure). Certain bonds (e.g., magnetic bonds) between atoms within these “stain” molecules absorb some or all light, including, but not limited to visible light. Therefore, light may not be reflected as much as it did prior to the presence, e.g., accumulation, of the stain molecule(s), and, consequently, the teeth appear darker or dark. These magnetic intramolecular bonds between atoms that absorb light are known as chromophore bonds.

Teeth first erupt into the mouth already having darker molecules (e.g., naturally occurring dark molecules) trapped within the tooth structure, left behind from the enamel and dentin growth process during tooth formation. Therefore, some people naturally have lighter teeth than others, even when teeth first erupt into the mouth. Such “pigment molecules” are already present within the tooth structure upon eruption into the mouth during childhood.

After teeth have erupted into the mouth, people may introduce dark molecules into their mouth, e.g., they consume foods and beverages that contain dark molecules. Common examples of food products that contain comparatively high concentrations of dark, staining molecules include coffee, red wine, cherries, tea, some sodas, blueberries, blackberries, pomegranates, some vinegars (e.g., balsamic), some spices (e.g., curry), etc. The accumulation of organic dark molecules on the surface of teeth during, or as a result of, eating and drinking, may be referred to as “extrinsic stain” and the organic dark molecules may be referred to as “stain molecules.”

Extrinsic stain molecules may start a slow process of soaking into tooth structure upon introduction into the mouth, e.g., immediately upon introduction into the mouth. Once inside the tooth structure (e.g., below the superficial surface of the tooth), the same stain molecules are referred to as “intrinsic stains.” Once inside the tooth structure, stain molecules may be difficult to remove (e.g., in some cases cannot be removed) by cleaning the surface of the tooth with brushing or even by professional teeth cleaning.

Stain molecules and pigment molecules may tend to attach (e.g., bind) together, thereby forming larger molecules inside teeth (e.g., within the tooth structure). Over time, intrinsic stains may pack deeper and tighter into the tooth structure, while simultaneously attaching together (e.g., aggregating). This results in a deep, tightly packed, tightly woven mass of organic stain material (e.g., collections of molecules bound together) throughout the microstructure of the teeth. The teeth may become darker as such stain molecules and pigment molecules collect over time. The crystalline microstructure of teeth may also become more “clogged-up” as stain molecules and pigment molecules fill in and clog up the spaces between the microscopic crystallites of the tooth structure.

Whitening of teeth may occur via two processes, which may occur simultaneously: oxygenation and conversion.

In the oxygenation process, breakdown products of hydrogen peroxide (e.g., ions, free radicals, and oxygen) bubble (e.g., aggressively and/or microscopically bubble) into the microstructure of the teeth. As these products, e.g., gases, form, they may start to break apart large organic stain mass(es) inside the tooth structure, resulting in smaller stain agglomerates/molecules. Aggressive microscopic bubbling inside the tooth microstructure can push some of these smaller stain molecules out of the tooth structure. This may reverse the process of an extrinsic stain becoming an intrinsic stain. Moreover, it may remove some of the mass of stain material (e.g., stain molecules and/or pigment molecules) in the tooth structure, advantageously causing the teeth to appear lighter in color. The bubbling may also result in some (e.g., partial) cleansing of the internal tooth microstructure (sometimes referred to as “conditioning” of the tooth structure).

In the conversion process, whitening products, e.g., peroxide based teeth whitening gels, may break down into various ions, radicals, and oxygen. Perhydroxyl radicals, a common byproduct of hydrogen peroxide, attack (e.g., specifically and/or selectively attack) the chromophore bonds within stain molecules and pigment molecules, breaking these bonds apart. As such, perhydroxyl radicals may induce the destruction of the specific intramolecular bonds that are responsible, e.g., at least partially responsible, for the absorption of visible light, and the resulting apparent darkening of the teeth. As chromophore bonds are broken, fewer light-absorbing chromophore bonds remain to absorb visible light. Therefore as chomophore bonds are destroyed, more visible light is reflected from the teeth, resulting in a lighter and/or brighter and/or whiter appearance of the teeth. Some chromophore bonds are stronger than other chromophore bonds and may therefore be more difficult to pull or break apart. Stronger chromophore bonds may therefore require higher concentrations of whitening products and/or chemicals (e.g., peroxide breakdown products) to be broken apart. As the chomophore bonds of the stain molecules and pigment molecules are broken, the size of the stain molecules and pigment molecules (and or agglomerates) decreases. As these stain molecules and/or pigment molecules decrease in size, they may be more easily removed from the microstructure of teeth by the oxygenation process discussed herein.

The teeth of a young teenager may whiten extremely well and very quickly. This is because the permanent adult teeth have not been in the mouth for very long. Such teeth have not had sufficient time to absorb significant amounts of stain or pigment, and thus the microstructure may still be clean. Breakdown products from peroxide can therefore soak into the teeth (which have not had a chance to “clog up”), e.g., very quickly in higher amounts, whitening the teeth well and/or effectively in a short amount of time. On the other hand, the teeth of older (e.g., geriatric) patients can be difficult to whiten (e.g., difficult or time consuming to whiten). This is because older teeth may be so heavily clogged with stain and or pigment molecules and/or agglomerates that they are less able to (if not incapable of) absorb the breakdown products from peroxide. Thus, older teeth are generally less capable or incapable of “quickly” absorbing the breakdown products, and will absorb the breakdown products only slowly, e.g., very slowly.

There have been several common ways to professionally whiten teeth with custom tooth whitening, including, but not necessarily limited to: daytime vital tray whitening, nighttime vital tray whitening, in-office power whitening, combination whitening, and Deep Bleaching. Daytime whitening techniques are generally understood to be those that are performed while the patient is awake, and nighttime whitening techniques are generally understood to be those that are performed while the patient is asleep (e.g., daytime and nighttime may not actually be correlated to conventional day and night, particularly for those people who work shifts requiring that they function at night and sleep during the day).

### Daytime Vital Tray Whitening

In daytime vital tray whitening, e.g., when the patient is awake, a thin, flexible tray may be created to fit over the teeth. A whitening gel (e.g., a hydrogen peroxide whitening gel) may be placed in the tray, and the tray containing the gel worn for 30-60 minutes. Alternatively, the tray may be worn for about 5-300 minutes, about 10-240 minutes, about 15-180 minutes, about 20-120 minutes, about 25-60 minutes, about 30-45 minutes, or any other length of time that is beneficial to the desired level or degree of whitening. The tray may be worn once a day, occasionally twice a day, occasionally three times a day, or any other number of times that is beneficial to the desired level of whitening.

Hydrogen peroxide may be selected as the whitening chemical for daytime vital tray whitening. This is because it breaks down quickly, releasing high amounts of peroxide breakdown products. Daytime whitening in trays may be limited by more frequent and aggressive swallowing that may be observed when the user (e.g., the patient wearing the tray(s)) is awake. Frequent and deeper swallowing may create a decreased pressure (e.g., a vacuum or a partial vacuum) inside the mouth when swallowing, resulting in sucking saliva into the whitening tray (e.g., into the tray with the whitening chemical, such as hydrogen peroxide) and/or sucking whitening gel out of the whitening tray. Saliva may have a comparatively high concentration of the natural antioxidant enzyme, glutathione peroxidase. Peroxidase destroys peroxide, e.g., destroys peroxide on contact. Peroxidase introduced into the tray, e.g., because of the more frequent and deeper swallowing observed when patients are awake, destroys (e.g., breaks down) the whitening gel, e.g., quickly. The short dwell time and/or active period of the whitening chemical is why hydrogen peroxide may be advantageously selected—it breaks down quickly. The faster the whitening chemical breaks down, the less the antioxidant effects of saliva affect the whitening process. Ideally, the whitening chemical will give off as much of the byproducts as possible before it is destroyed by peroxidase in the saliva.

There are few ways, e.g., no truly effective ways, to protect gums with tray whitening. Therefore, particularly high concentrations of hydrogen peroxide may be disadvantageous or undesirable, e.g., in some instances may not be used: high concentration hydrogen peroxide may chemically burn the gums. Time may therefore be limited (e.g., very limited) with daytime whitening, which may not allow sufficient time for the oxygenation phase of whitening to cleanse tooth structure internally.

Whitening trays may be fabricated in a wide range of qualities of fit. Some whitening trays fit loosely, allowing ingress (e.g., rapid ingress) of damaging salivary peroxidase more quickly. Well-fitting trays help seal out saliva. However frequent and deep swallowing may create sufficiently decreased pressure in the mouth (e.g., such a degree of vacuum) such that even well-fitting whitening trays cannot effectively prevent ingress of damaging saliva (e.g., to allow extended periods of uninterrupted whitening).

Given the limited concentration of whitening chemicals (e.g., hydrogen peroxide) that may comfortably be used in whitening trays, and given the short period of chemical activity (e.g., prior to saliva-induced deactivation or destruction), there may not be sufficient time to thoroughly cleanse the internal of the tooth microstructure such that higher volumes of peroxide breakdown products may effectively enter the tooth structure. Moreover, there may be insufficient concentration of whitening gel to pull the stronger chromophore bonds apart. As discussed herein, carbamide peroxide generally breaks down slower than other commonly used whitening products and consequently may generate a lower concentration of breakdown products than faster-degrading whitening products, e.g., hydrogen peroxide. Therefore, carbamide peroxide may not have or generate sufficient concentrations of breakdown products due to its slower breakdown. Hydrogen peroxide breaks down faster than carbamide peroxide and consequently generates a higher concentration of break down products than carbamide peroxide. Therefore, hydrogen peroxide may be more effective at breaking apart chromophore bonds than carbamide peroxide, particularly under the conditions imposed by at home daytime whitening. Regardless, there may be cases in which insufficient whitening gel (e.g., hydrogen peroxide) may be present to break down to generate sufficient break down products to pull apart the strongest chromophore bonds.

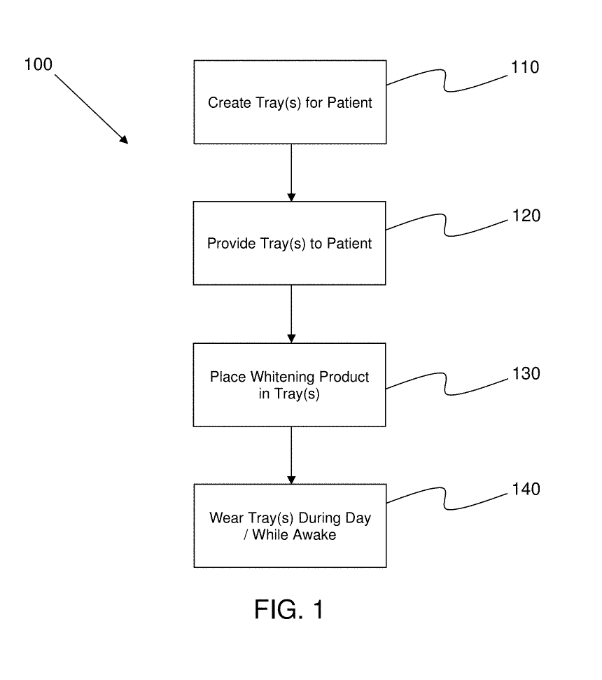

FIG. 1 shows a flow chart of one embodiment of a method for daytime vital tray whitening of teeth 100. At step one 110, a thin, flexible tray may be created to fit over the patient's teeth. The thin, flexible tray may be a maxillary tray. The thin, flexible tray may be a mandibular tray. At step two 120, the tray created in step one 110, including one or more of a maxillary tray and a mandibular tray, are provided to the patient.

The patient may be instructed regarding one or more steps of the method for daytime vital tray whitening of teeth 100. Furthermore, the patient may be provided one or more products, e.g., whitening products, in addition to the trays.

At step three 130, the whitening product is placed in the tray. The whitening product may be placed in the tray by the patient, a member of the patient's family, a caretaker, or a dental professional, or any other person capable of placing the whitening product in the tray. In some embodiments, the whitening product is a whitening gel. In some embodiments, the whitening product is a hydrogen peroxide whitening gel. In some embodiments, the whitening product is a hydrogen peroxide whitening gel having a concentration of less than about 50%, less than about 45%, less than about 40%, less than about 35%, less than about 30%, less than about 25%, less than about 20%, less than about 17.5%, less than about 15%, less than about 12.5%, less than about 10%, less than about 9%, less than about 8%, less than about 7%, less than about 6%, less than about 5%, less than about 4%, less than about 3%, less than about 2%, or less than about 1%. In some embodiments, the whitening product is a hydrogen peroxide whitening gel having a concentration of between about 7-50%, between about 8-45%, between about 9-40%, between about 10-35%, between about 11-30%, between about 12-25%, between about 13-20%, or between about 14-17%. In some embodiments, the whitening product is a hydrogen peroxide whitening gel having a concentration of between about 3-20%, between about 4-19%, between about 5-18%, between about 6-17%, between about 7-16%, between about 8-15%, between about 9-14%, between about 10-13%, or between about 11-12%. In some embodiments, the whitening product is a hydrogen peroxide whitening gel having a concentration of between about 5.5-6%. In some embodiments, the whitening product is a chemically accelerated hydrogen peroxide whitening gel. In some embodiments, the whitening product is a hydrogen peroxide whitening gel having a concentration as high as possible without damaging the oral structures or mucosa or causing pain to the patient. In some embodiments, the whitening product is a whitening gel comprising a chemical that breaks down quickly into whitening products.

At step four 140, the tray containing the whitening product is placed in the mouth and worn for a time period during the daytime, e.g., while awake. In some embodiments, the tray containing the whitening product is worn for less than about 180 minutes, less than about 170 minutes, less than about 160 minutes, less than about 150 minutes, less than about 140 minutes, less than about 130 minutes, less than about 120 minutes, less than about 110 minutes, less than about 100 minutes, less than about 90 minutes, less than about 80 minutes, less than about 70 minutes, less than about 60 minutes, less than about 50 minutes, less than about 40 minutes, less than about 30 minutes, less than about 20 minutes, less than about 10 minutes, or less than about 5 minutes. In some embodiments, the tray containing the whitening product is worn for between about 5-300 minutes, about 10-240 minutes, about 15-180 minutes, about 20-120 minutes, about 25-60 minutes, about 30-45 minutes, or any other length of time that is beneficial to the desired level or degree of whitening.

In some embodiments of the method for daytime vital tray whitening of teeth 100, steps three 130 and four 140 may be performed more than once (e.g., repeated) during a single day. In some embodiments, in a single day steps three 130 and four 140 are performed 2 times, 3 times, 4 times, 5 times, 6 times, 7 times, 8 times, 9 times, or 10 times.

The method for daytime vital tray whitening of teeth 100 may be carried out for any number of days, e.g., consecutive days, or non-consecutive days over a time period. For example, the method for daytime vital tray whitening of teeth 100 may be carried out for 1 day, 2, days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 24 days, 26 days, 28 days, 30 days, 32 days, 34 days, 36 days, 38 days, or 40 days. In some embodiments, the method for daytime vital tray whitening of teeth 100 is carried out for more than 40 days. In some embodiments, the method for daytime vital tray whitening of teeth 100 may be carried out for less than about 18 months, less than about 17 months, less than about 16 months, less than about 15 months, less than about 14 months, less than about 13 months, less than about 12 months, less than about 11 months, less than about 10 months, less than about 9 months, less than about 8 months, less than about 7 months, less than about 6 months, less than about 5 months, less than about 4 months, less than about 3 months, less than about 2 months, or less than about 1 month. In some embodiments, the method for daytime vital tray whitening of teeth 100 is carried out until the teeth reach a desired shade, e.g., lighten to a desired shade.

The method for daytime vital tray whitening of teeth 100, or individual steps thereof, may be carried out until the teeth have lightened by about 5%. In some embodiments, the method for daytime vital tray whitening of teeth 100, or individual steps thereof, may be carried out until the teeth have lightened by about 1%, about 2%, about 3%, about 4%, about 5%, about 6%, about 7%, about 8%, about 9%, about 10%, about 12%, about 14%, about 16%, about 18%, about 20%, about 22%, about 24%, about 26%, about 28%, about o30%, about 35%, about 40%, about 45%, about 50%, about 55%, about 60%, about 65%, about 70%, or about 75%. Percentage of lightening may be based on a scale of the shade of the tooth to begin with by comparison to white. Alternatively, percentage of lightening may be based on a scale of black to white. In some embodiments, the method for daytime vital tray whitening of teeth 100, or individual steps thereof, may be carried out until the teeth have lightened by 1 shade, 2 shades, 3 shades, 4 shades, 5 shades, 6 shades, 7 shades, 8 shades 9 shades, 10 shades, 11 shades, 12 shades, 13 shades, 14 shades, 15 shades, 16 shades, 17 shades, 18 shades, 19 shades, 20 shades, 25, shades, 30 shades, 35 shades, 40 shades, 45 shades, 50 shades, 55 shades, 60 shades, 65 shades, 70 shades, 75 shades, or 80 shades. In some embodiments, the desired lightening of the teeth (using either percentage or shade) may be achieved using one or more iteration of the method, or individual steps thereof. For example, the desired lightening of the teeth (using either percentage or shade) may be achieved by performing the method, or individual steps thereof, 1 time, 2 times, 3 times, 4 times, 5 times, 6 times, 7 times, 8 times 9 times, 10 times, 15 times or less, 20 times or less, 25 times or less, 30 times or less, 35 times or less, 40 times or less, 45 times or less, 50 times or less, 60 times or less, 70 times or less, 80 times or less, 90 times or less, 100 times or less, 110 times or less, 120 times or less, 130 times or less, 140 times or less, 150 times or less, 160 times or less, 170 times or less, 180 times or less, 190 times or less, 200 times or less, 210 times or less 220 times or less, 230 times or less, 240 times or less, 250 times or less, 260 times or less, 270 times or less, 280 times or less, 290 times or less, 300 times or less, 310 times or less, 320 times or less, 330 times or less, 340 times or less, 350 times or less, 360 times or less, 370 times or less, 380 times or less, 390 times or less, or 400 times or less.

### Nighttime Vital Tray Whitening

In nighttime vital tray whitening, whitening trays may be fabricated in the same way that daytime whitening trays are fabricated. These trays may be filled with whitening gel and inserted over the teeth prior to sleep, e.g., immediately prior to sleep. When lying down to sleep, salivation production naturally slows. Additionally, swallowing frequency and depth may diminish. When a patient actually falls asleep, the rate of swallowing may drop nearly to zero. Such decreased saliva production and reduction in swallowing may result in an unpleasant odor of the breath following an extended period of sleep (e.g., a night's sleep). Salivary flow and swallowing are processes used by the mouth to self-cleanse itself. When the self-cleansing of salivating and swallowing stops (i.e., during the night, when asleep), bacteria are able to grow relatively undisturbed—increased bacterial growth is responsible for the unpleasant odor that may be experienced after a night's sleep, often referred to as “morning breath.”

When whitening tray fit is poor (e.g., when the trays fit loosely) saliva may still enter the whitening trays somewhat quickly. As discussed herein, certain saliva constituents may break down whitening chemicals. Therefore, poor tray fitment may result in rapid destruction of the whitening gel, even in sleeping patients who have decreased saliva production and reduced rates and depth of swallowing. However, whitening trays that fit properly and seal against the gum line areas of the teeth may advantageously prevent the damaging salivary peroxidase from entering the whitening trays.

Carbamide peroxide is a common nighttime whitening chemical. Carbamide peroxide breaks down more slowly than hydrogen peroxide. In well-fitting trays, carbamide peroxide may be active for more than 6 hours, with some level of activity extending through 10 hours. However, due to its slower break-down rate, carbamide peroxide generates a lower concentration of breakdown products over time.

When breakdown products (e.g., ions, free radicals, and oxygen) are given off over a longer period of time, at any given time there are generally less or fewer breakdown products present to enter the teeth (and that are entering the teeth) at any given time. However, a positive side-effect of protracted break-down is the prolonged cleansing ability of the oxygenation phase. Throughout the night (e.g., the entire time the patient is asleep), the teeth's microstructure becomes increasingly cleansed. Though significant cleansing of the internal structure of the teeth may be accomplished, there is generally an insufficient concentration of peroxide breakdown products (e.g., due to the slow breakdown process of carbamide peroxide) to pull apart stronger chromophore bonds (e.g., stronger stains).

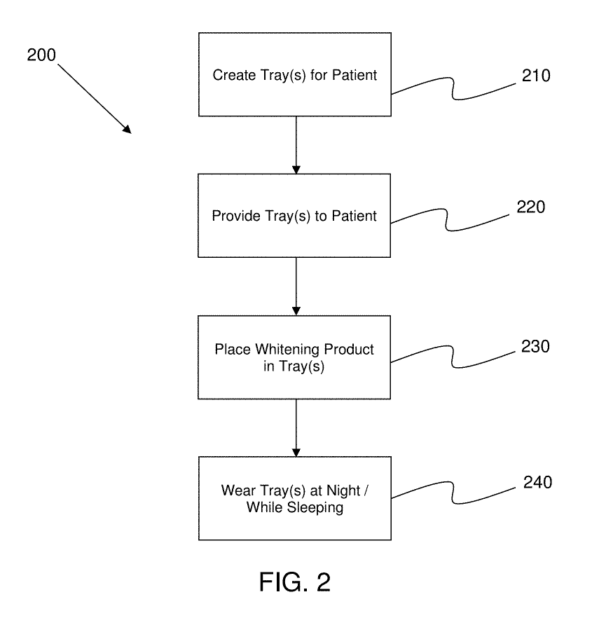

FIG. 2 shows a flow chart of one embodiment of a method for nighttime vital tray whitening of teeth 200. At step one 210 a tray may be created to fit over the patient's teeth. The tray may be a maxillary tray. The tray may be a mandibular tray. At step 220, the tray created in step one 210, including one or more of a maxillary tray and a mandibular tray, is provided to the patient.

The patient may be instructed regarding one or more steps of the method for nighttime vital tray whitening of teeth 200. Furthermore, the patient may be provided one or more products, e.g., whitening products, in addition to the trays.

At step three 230, the whitening product is placed in the tray. The whitening product may be placed in the tray by the patient, a member of the patient's family, a caretaker, or a dental professional, or any other person capable of placing the whitening product in the tray. In some embodiments, the whitening product is a whitening gel. In some embodiments, the whitening product is a carbamide peroxide whitening gel. In some embodiments, the whitening product is a carbamide peroxide whitening gel having a concentration of less than about 70%., less than about 65%, less than about 60%, less than about 55%, less than about 50%, less than about 45%, less than about 40%, less than about 35%, less than about 30%, less than about 25%, less than about 20%, less than about 17.5%, less than about 15%, less than about 12.5%, less than about 10%, less than about 9%, less than about 8%, less than about 7%, less than about 6%, less than about 5%, less than about 4%, less than about 3%, or less than about 2%. In some embodiments, the whitening product is a carbamide peroxide whitening gel having a concentration of between about 7-50%, between about 8-45%, between about 9-40%, between about 10-35%, between about 11-30%, between about 12-25%, between about 13-20%, or between about 14-17%. In some embodiments, the whitening product is a carbamide peroxide whitening gel having a concentration of between about 10-70%, between about 11-65%, between about 12-60%, between about 13-55%, between about 14-50%, between about 15-45%, between about 16-40%, between about 17-35%, between about 18-30%, or between about 19-25%. In some embodiments, the whitening product is a chemically accelerated carbamide peroxide whitening gel. In some embodiments, the whitening product is a carbamide peroxide whitening gel having a concentration as high as possible without damaging the oral structures or mucosa or causing pain to the patient. In some embodiments, the whitening product is a whitening gel comprising a chemical that breaks down slowly into whitening products. In some embodiments, the whitening product is a whitening gel comprising a chemical that breaks down slowly enough so as to remain active (e.g., maintain whitening activity) for at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, or at least about 24 hours. In some embodiments, the whitening product is a whitening gel comprising a chemical that breaks down slowly enough so as to remain active (e.g., maintain whitening activity) for between about 2-24 hours, between about 3-22 hours, between about 4-20 hours, between about 5-18 hours, between about 6-16 hours, between about 7-14 hours, between about 8-12 hours, or between about 9-11 hours.

At step four 240, the tray containing the whitening product is placed in the mouth and worn for a time period during the nighttime, e.g., while asleep. The whitening product may be placed in the tray and then in tray placed in the mouth shortly before, e.g., immediately before, the patient goes to bed. In some embodiments, the tray containing the whitening product is worn for the entire time the patient is asleep, e.g., about 6-8 hours. In some embodiments, the tray containing the whitening product is worn for less than about 12 hours, less than about 11.5 hours, less than about 11 hours, less than about 10.5 hours, less than about 10 hours, less than about 9.5 hours, less than about 9 hours, less than about 8.75 hours, less than about 8.5 hours, less than about 8.25 hours, less than about 8 hours, less than about less than about 7.75 hours, less than about 7.5 hours, less than about 7.25 hours, less than about 7 hours, less than about 6.75 hours, less than about 6.5 hours, less than about 6.25 hours, less than about 6 hours, less than about 5.75 hours, less than about 5.5 hours, less than about 5.25 hours, less than about 5 hours, less than about 4.75 hours, less than about 4.5 hours, less than about 4.25 hours, less than about 4 hours, less than about 3.75 hours, less than about 3.5 hours, less than about 3.25 hours, less than about 3 hours, less than about 2.75 hours, less than about 2.5 hours, less than about 2.25 hours, less than about 2 hours, less than about 1.75 hours, less than about 1.5 hours, less than about 1.25 hours, or as little as about 1 hour. In some embodiments, the tray containing the whitening product is worn for between about 2-20 hours, between about 2.25-19.5 hours, between about 2.5-19 hours, between about 2.75-18.5 hours, between about 3-18 hours, between about 3.25-17.5 hours, between about 3.5-17 hours, between about 3.75-16.5 hours, between about 4-16 hours, between about 4.25-15.5 hours, between about 4.5-15 hours, between about 4.75-14.5 hours, between about 5-14 hours, between about 5.25-13.5 hours, between about 5.5-13 hours, between about 5.75-12.5 hours, between about 6-11.5 hours, between about 6.25-11 hours, between about 6.5-10.5 hours, between about 6.75-10 hours, between about 7-9.5 hours, between about 7.25-9 hours, between about 7.5-8.5 hours, or between about 7.75-8 hours, or any other length of time that is beneficial to the desired level or degree of whitening.

The method for nighttime vital tray whitening of teeth 200 may be carried out for any number of nights, e.g., consecutive nights, or non-consecutive nights over a time period. For example, the method for nighttime vital tray whitening of teeth 200 may be carried out for 1 night, 2, nights, 3 nights, 4 nights, 5 nights, 6 nights, 7 nights, 8 nights, 9 nights, 10 nights, 11 nights, 12 nights, 13 nights, 14 nights, 15 nights, 16 nights, 17 nights, 18 nights, 19 nights, 20 nights, 21 nights, 22 nights, 24 nights, 26 nights, 28 nights, 30 nights, 32 nights, 34 nights, 36 nights, 38 nights, 40 nights, 45 nights, 50 nights, 55 nights, 60 nights, 65 nights, 70 nights, 75 nights, 80 nights, 85 nights, 90 nights 95 nights, 100 nights, 105 nights, 110 nights, 115 nights, 120 nights, 125 nights, 130 nights, 135 nights, 140 nights, 145 nights, 150 nights, 155 nights, 160 nights, 170 nights, 180 nights, 190 nights, 200 nights, 210 nights, 220 nights, 230 nights, 240 nights, 250 nights, 260 nights, 270 nights, 280 nights, 290 nights, 300 nights, 310 nights, 320 nights, 330 nights, 340 nights, 350 nights, 360 nights, or more nights. In some embodiments, the method for nighttime vital tray whitening of teeth 200 is carried out for more than 40 nights. In some embodiments, the method for nighttime vital tray whitening of teeth 200 is carried out for less than about 24 months, less than about 23 months, less than about 22 months, less than about 21 months, less than about 20 months, less than about 19 months, less than about 18 months, less than about 17 months, less than about 16 months, less than about 15 months, less than about 14 months, less than about 13 months, less than about 12 months, less than about 11 months, less than about 10 months, less than about 9 months, less than about 8 months, less than about 7 months, less than about 6 months, less than about 5 months, less than about 4 months, less than about 3 months, or less than about 2 months. In some embodiments, the method for nighttime vital tray whitening of teeth 200 is carried out until the teeth reach a desired shade, e.g., lighten to a desired shade.

The method for nighttime vital tray whitening of teeth 200, or individual steps thereof, may be carried out until the teeth have lightened by about 5%. In some embodiments, the method for nighttime vital tray whitening of teeth 200, or individual steps thereof, may be carried out until the teeth have lightened by about 1%, about 2%, about 3%, about 4%, about 5%, about 6%, about 7%, about 8%, about 9%, about 10%, about 12%, about 14%, about 16%, about 18%, about 20%, about 22%, about 24%, about 26%, about 28%, about 30%, about 35%, about 40%, about 45%, about 50%, about 55%, about 60%, about 65%, about 70%, or about 75%. In some embodiments, the method for nighttime vital tray whitening of teeth 200, or individual steps thereof, may be carried out until the teeth have lightened by 1 shade, 2 shades, 3 shades, 4 shades, 5 shades, 6 shades, 7 shades, 8 shades 9 shades, 10 shades, 11 shades, 12 shades, 13 shades, 14 shades, or 15 shades, 16 shades, 17 shades, 18 shades, 19 shades, 20 shades, 25, shades, 30 shades, 35 shades, 40 shades, 45 shades, 50 shades, 55 shades, 60 shades, 65 shades, 70 shades, 75 shades, or 80 shades. In some embodiments, the desired lightening of the teeth (using either percentage or shade) may be achieved using one or more iteration of the method, or individual steps thereof. For example, the desired lightening of the teeth (using either percentage or shade) may be achieved by performing the method, or individual steps thereof, 1 time, 2 times, 3 times, 4 times, 5 times, 6 times, 7 times, 8 times 9 times, 10 times, 15 times or less, 20 times or less, 25 times or less, 30 times or less, 35 times or less, 40 times or less, 45 times or less, 50 times or less, 60 times or less, 70 times or less, 80 times or less, 90 times or less, 100 times or less, 110 times or less, 120 times or less, 130 times or less, 140 times or less, 150 times or less, 160 times or less, 170 times or less, 180 times or less, 190 times or less, 200 times or less, 210 times or less 220 times or less, 230 times or less, 240 times or less, 250 times or less, 260 times or less, 270 times or less, 280 times or less, 290 times or less, 300 times or less, 310 times or less, 320 times or less, 330 times or less, 340 times or less, 350 times or less, 360 times or less, 370 times or less, 380 times or less, 390 times or less, or 400 times or less.

### In-Office Power Whitening

In-office power whitening may allow higher concentrations of whitening chemicals to be used because it is performed by a whitening professional. Retractor appliances may be inserted into the mouth to hold the tongue, cheeks, and/or lips away from the teeth. Then, a barrier material may be placed over the gums, thereby reducing or preventing a whitening chemical, such as hydrogen peroxide gel, from coming into contact with the gums. Because of this, higher concentration whitening gel may be used. If whitening gels having such concentrations of whitening chemicals were to come into contact with cheeks, lips, gums or any other soft tissues, those tissues could be chemically burned, which may cause pain to the patient.

After isolating the teeth, high concentration hydrogen peroxide (or other whitening chemical) may be applied to the facial surfaces (visible surfaces) of the teeth, e.g., the surfaces of the teeth that can be seen when smiling. Though hydrogen peroxide breaks down faster, e.g., significantly faster, than carbamide peroxide, for various reasons it may be advantageous for the peroxide gel break down even faster. For example, visits in the dental office may desirably be limited in time—the longer the time, the more cost for the patient. Also, patients may become anxious and uncomfortable when in a dental chair for long periods of time. Therefore, dental office whitening sessions may advantageously be limited to 15-60 minutes of contact time of the high concentration hydrogen peroxide gel with the teeth. However, other durations of contact may be used, including, but not limited to about 5-180 minutes, about 10-150 minutes, about 15-120 minutes, about 20-90 minutes, about 25-60 minutes, about 30 minutes, or any other length of time that is beneficial to the desired level of whitening. In view of the above, it may be desirable for the whitening chemical (e.g., hydrogen peroxide) to break down, giving off its “bleaching factors” (including, but not limited to, free radicals and ions) as quickly as possible. Therefore, high concentration hydrogen peroxide power whitening gels may be chemically activated, e.g., accelerated, with a chemical catalyst. Such acceleration may involve separating the gel mixture (e.g., the accelerator and the whitening gel) into at least two separate containers, e.g., a dual barrel syringe, and mixing them immediately prior to application to the teeth.

In-office power whitening may be performed with a photon energy source (e.g., a strong light or laser). However, some clinical studies have suggested that the use of a photon energy source has little beneficial effect on the whitening process while potentially causing significant inflammation and discomfort to the tooth pulp (nerve). Yet, for various reasons, such “enhanced” whitening processes (e.g., incorporating the use of whitening lights and lasers) are offered and/or endorsed by many companies/providers: for example, some companies/providers believe that these “enhanced” whitening processes and/or services are beneficial for marketing purposes and consumer perception.

Regardless of the potentially high concentrations that may be used, the contact time between the whitening gel and the teeth of in-office whitening may be insufficient to result (e.g., routinely result) in significant and/or lasting whiteness. The contact time may not be sufficient to create/allow significant oxygenation cleansing of the tooth microstructure. Regardless of how active and high concentration of a chemically activated whitening gel, if the whitening chemical(s) and/or their breakdown products are unable to penetrate the microstructure of the teeth (e.g., penetrate deeply/well), little whitening may be accomplished.

After an in-office whitening visit, it is not uncommon for the teeth to be in a similar condition (in terms of whiteness) (e.g., a similar or virtually the same condition or shade) as prior to the dental office visit. Some in-office visits may yield some minor apparent success. However, such observed whitening is frequently short-lived. In-office power whitening may result in the temporary dehydration of teeth, which may, result in temporary appearance of whiter teeth. But, within a short time (e.g., one day, two days, three days, four days, etc.), the teeth may rehydrate and return to their pre-dehydration shade (e.g., become darker again).

As will be understood, in-office whitening may be unpredictable. Due to such unpredictability, patients may be instructed to continue the whitening process at home, after an in-office power whitening, with whitening trays for daytime and/or nighttime whitening. In-office whitening followed by daytime and/or nighttime whitening is sometimes referred to as “combination whitening.”

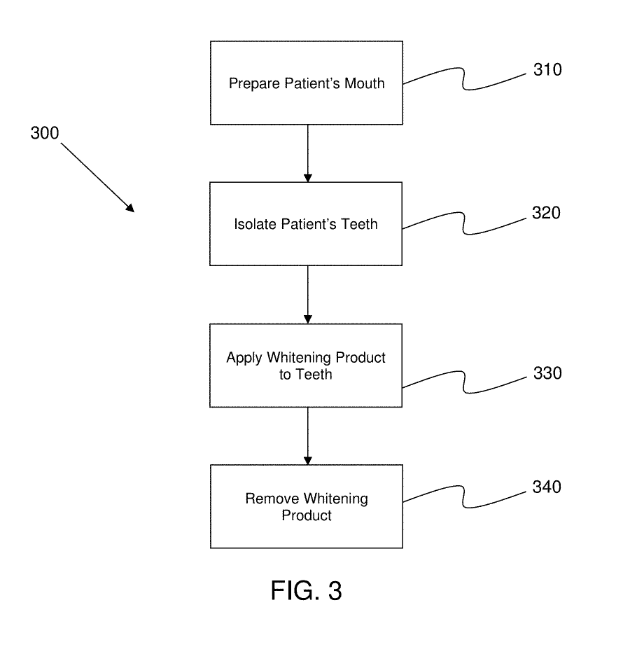

FIG. 3 shows a flow chart of one embodiment of a method for in-office power whitening of teeth 300. At step one 310 the patient's mouth may be prepared. For example, as discussed herein retractor appliances may be inserted into the mouth to hold the tongue, cheeks, and/or lips away from the teeth. Holding one or more of the tongue, cheeks, and/or lips away from the teeth may help prevent whitening gels and/or chemicals from contacting these soft tissues while acting on the teeth.

At step two 320, the patients teeth may be isolated. For example, a barrier material may be placed over the gum to reduce or prevent contact of the whitening gel or chemicals with the gums. Generally, in-office power whitening uses whitening products having higher concentrations of whitening chemicals, e.g., hydrogen peroxide. Therefore, it may be advantageous to keep these high-concentration chemicals away from the patient's soft tissues or oral mucosa to prevent chemical burning and/or pain. In some embodiments, the whitening product used in in-office power whitening comprises hydrogen peroxide. In some embodiments, the whitening product used in in-office power whitening comprises chemically accelerated hydrogen peroxide. In some embodiments, the whitening product used in in-office power whitening comprises a mixture of hydrogen peroxide and carbamide peroxide. In some embodiments, the whitening product used in in-office power whitening comprises a mixture of chemically accelerated hydrogen peroxide and chemically accelerated carbamide peroxide. In some embodiments, the whitening product used in in-office power whitening comprises carbamide peroxide. In some embodiments, the whitening product used in in-office power whitening comprises chemically accelerated carbamide peroxide.

At step three 330, whitening product is applied to the patient's teeth, e.g., the facial surface of the teeth. In some embodiments, the whitening product is a whitening gel. In some embodiments, the whitening product is a hydrogen peroxide whitening gel. In some embodiments, the whitening product is a hydrogen peroxide whitening gel having a concentration of less than about 60%, less than about 55%, less than about 50%, less than about 45%, less than about 40%, less than about 35%, less than about 30%, less than about 25%, less than about 20%, less than about 17.5%, less than about 15%, less than about 12.5%, less than about 10%, less than about 9%, less than about 8%, less than about 7%, less than about 6%, less than about 5%, less than about 4%, less than about 3%, or less than about 2%. In some embodiments, the whitening product is a hydrogen peroxide whitening gel having a concentration of between about 7-50%, between about 8-45%, between about 9-40%, between about 10-35%, between about 11-30%, between about 12-25%, between about 13-20%, or between about 14-17%. In some embodiments, the whitening product is a chemically accelerated whitening gel, e.g., the whitening product comprises a catalyst to accelerate breakdown of the whitening chemicals, e.g., to even more quickly form reactive species. In some embodiments, the whitening product is a whitening gel comprising a chemical that breaks down quickly into whitening products.

The whitening product is left on the patient's teeth for some amount of time. As in-office power whitening is generally done in a whitening or dental professional's office, rather than in the comfort of a patient's own home, the product may left on the patient's teeth for a comparatively short time. In some embodiments, the whitening product is left on the patient's teeth for less than about 180 minutes, less than about 170 minutes, less than about 160 minutes, less than about 150 minutes, less than about 140 minutes, less than about 130 minutes, less than about 120 minutes, less than about 110 minutes, less than about 100 minutes, less than about 95 minutes, less than about 90 minutes, less than about 85 minutes, less than about 80 minutes, less than about 75 minutes, less than about 70 minutes, less than about 65 minutes, less than about 60 minutes, less than about 55 minutes, less than about 50 minutes, less than about 45 minutes, less than about 40 minutes, less than about 35 minutes, less than about 30 minutes, less than about 25 minutes, less than about 20 minutes, less than about 15 minutes, less than about 10 minutes, less than about 9 minutes, less than about 8 minutes, less than about 7 minutes, less than about 6 minutes, less than about 5 minutes, less than about 4 minutes, less than about 3 minutes, or less than about 2 minutes. In some embodiments, the whitening product is left on the patient's teeth for between about 15-60 minutes. However, other durations of contact may be used, including, but not limited to about 5-180 minutes, about 10-150 minutes, about 15-120 minutes, about 20-90 minutes, about 25-60 minutes, about 30 minutes, or any other length of time that is beneficial to the desired level of whitening. In some embodiments, the whitening product is left on the patient's teeth until the teeth reach a desired shade, e.g., lighten to a desired shade.

After the whitening product has been applied to the patient's teeth and left on the teeth for the desired amount of time, e.g., until the teeth have lightening to a desired shade, the whitening product may be removed from the patient's teeth and or the patient's mouth. In some embodiments, the patient's teeth may be rinsed and the patient's mouth flushed to remove any remaining or excess whitening product. This may be particularly advantageous when whitening products having high or very high concentrations of whitening chemicals or accelerants are used.

The method for in-office power whitening of teeth 300, or individual steps thereof, may be carried out until the teeth have lightened by about 5%. In some embodiments, the method for in-office power whitening of teeth 300, or individual steps thereof, may be carried out until the teeth have lightened by about 1%, about 2%, about 3%, about 4%, about 5%, about 6%, about 7%, about 8%, about 9%, about 10%, about 12%, about 14%, about 16%, about 18%, about 20%, about 22%, about 24%, about 26%, about 28%, about 30%, about 35%, about 40%, about 45%, about 50%, about 55%, about 60%, about 65%, about 70%, or about 75%. In some embodiments, the method for in-office power whitening of teeth 300, or individual steps thereof, may be carried out until the teeth have lightened by 1 shade, 2 shades, 3 shades, 4 shades, 5 shades, 6 shades, 7 shades, 8 shades 9 shades, 10 shades, 11 shades, 12 shades, 13 shades, 14 shades, or 15 shades, 16 shades, 17 shades, 18 shades, 19 shades, 20 shades, 25, shades, 30 shades, 35 shades, 40 shades, 45 shades, 50 shades, 55 shades, 60 shades, 65 shades, 70 shades, 75 shades, or 80 shades. In some embodiments, the desired lightening of the teeth (using either percentage or shade) may be achieved using one or more iteration of the method, or individual steps thereof. For example, the desired lightening of the teeth (using either percentage or shade) may be achieved by performing the method, or individual steps thereof, 1 time, 2 times, 3 times, 4 times, 5 times, 6 times, 7 times, 8 times 9 times, or 10 times.

### Combination Whitening

In combination whitening, as briefly discussed above, in-office power whitening is first performed/accomplished. Then, the patient is provided with the necessary tools and or products and instructed to perform daytime and/or nighttime vital tray whitening as explained elsewhere herein. While the results of combination whitening may, in some cases, be better (e.g., result in whiter teeth for longer periods of time), the process suffers from some limitations. As discussed above, e.g., in connection with daytime vital tray whitening, combination whitening may suffer from poor performance due to the limited concentration whitening products or gels that can be used in whitening trays (e.g., without chemically burning the patient's oral mucosa), and the short effective time period of chemical activity, there may be (e.g., generally will be) insufficient time to thoroughly cleanse the internal tooth microstructure. Therefore, high volumes of breakdown products (e.g., peroxide breakdown products) are unable to enter the tooth structure, e.g., the tooth internal microstructure, and insufficient concentration of whitening gel chemicals are present to break (e.g., pull apart) any stronger and/or darkening chromophore bonds. Ultimately, though cleansing of the internal structure of the teeth may be achieved, there is generally an insufficient concentration of peroxide breakdown products to pull apart (e.g., break) stronger chromophore bonds, e.g., any, sufficient, or all chomophore bonds, (stronger stains) that may be present. Therefore, the teeth may remain with a darker or dark appearance, or even not as light as the patient desires.

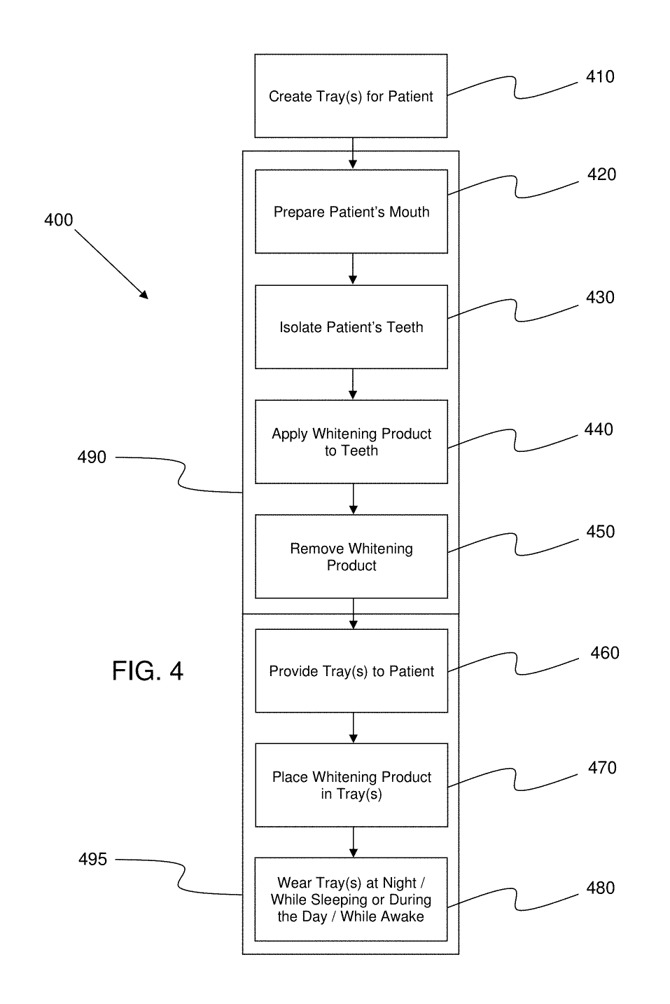

FIG. 4 shows a flow chart of one embodiment of a method for combination whitening of teeth 400. The method for combination whitening of teeth 400 may comprise an in-office power whitening portion 490 and an at home whitening portion 495.

At step one 410, trays may be created for the patient. The trays created for the patient may be similar if not the same as the trays created for the method for daytime vital tray whitening of teeth 100 or the method for nighttime vital tray whitening of teeth 200.

The in-office power whitening portion 490 comprises steps two 420 in which the patient's mouth is prepared, three 430 in which the patient's teeth are isolated, four 440 in which whitening product is applied to the patient's teeth, and five 450 in which the whitening product is removed from the patient's teeth. The steps of the method for combination whitening of teeth 400 in the in-office power whitening portion 490 may correspond to the steps of the method for in-office power whitening 300 discussed herein. For example: step two 420 (in which the patient's mouth is prepared) of method 400 may correspond to step one 310 (in which the patient's mouth is prepared) of method 300; step three 430 (in which the patient's teeth are isolated) of method 400 may correspond to step two 320 (in which the patient's teeth are isolated) of method 300; step four 440 (in which the whitening product is applied to the patient's teeth) of method 400 may correspond to step three 330 (in which the whitening product is applied to the patient's teeth) of method 300; and step five 450 (in which the whitening product is removed) may correspond to step four 340 (in which the whitening product is removed) of method 300. Some steps in method 400 may correspond to, be similar to, or even be identical to steps in method 300. While some steps in method 400 may correspond to steps in method 300, such correspondence is not required.

The at home whitening portion 495 of the method for combination whitening of teeth 400 comprises steps six 460 in which the trays created in step one 410 are provided to the patient, seven 470 in which whitening product is placed in the trays, and step eight in which the trays containing the whitening product are worn by the patient. The at home whitening portion 495 may be similar, if not identical to either or both of the method for daytime vital tray whitening of teeth 100 or the method for nighttime vital tray whitening of teeth 200. For example, the at home whitening portion 495 may comprise a nighttime whitening protocol, similar to the method for nighttime vital tray whitening of teeth 200. For example the at home whitening portion 495 may comprise a daytime whitening protocol, similar to the method for daytime vital tray whitening of teeth 100. For example, the at home whitening portion 495 may comprise both a daytime whitening protocol, similar to the method for daytime vital tray whitening of teeth 100, and a nighttime whitening protocol, similar to the method for nighttime vital tray whitening of teeth 200. For example: step six 460 (in which the trays created in step one 410 are provided to the patient) may correspond to one or both of step two 120 of method 100 and/or step two 220 of method 200; step seven 470 (470 in which whitening product is placed in the trays) may correspond to one or both of step three 130 of method 100 and/or step three 230 of method 200; and step eight 480 (in which the trays containing the whitening product are worn by the patient) may correspond to one or both of step four 140 of method 100 and/or step four 240 of method 200.

The method for combination whitening of teeth 400, or individual steps thereof, may be carried out until the teeth have lightened by about 5%. In some embodiments, the method for combination whitening of teeth 400, or individual steps thereof, may be carried out until the teeth have lightened by about 1%, about 2%, about 3%, about 4%, about 5%, about 6%, about 7%, about 8%, about 9%, about 10%, about 12%, about 14%, about 16%, about 18%, about 20%, about 22%, about 24%, about 26%, about 28%, about 30%, about 35%, about 40%, about 45%, about 50%, about 55%, about 60%, about 65%, about 70%, or about 75%. In some embodiments, the method for combination whitening of teeth 400, or individual steps thereof, may be carried out until the teeth have lightened by 1 shade, 2 shades, 3 shades, 4 shades, 5 shades, 6 shades, 7 shades, 8 shades 9 shades, 10 shades, 11 shades, 12 shades, 13 shades, 14 shades, or 15 shades, 16 shades, 17 shades, 18 shades, 19 shades, 20 shades, 25, shades, 30 shades, 35 shades, 40 shades, 45 shades, 50 shades, 55 shades, 60 shades, 65 shades, 70 shades, 75 shades, or 80 shades. In some embodiments, the desired lightening of the teeth (using either percentage or shade) may be achieved using one or more iteration of the method, or individual steps thereof. For example, the desired lightening of the teeth (using either percentage or shade) may be achieved by performing the method, or individual steps thereof, 1 time, 2 times, 3 times, 4 times, 5 times, 6 times, 7 times, 8 times 9 times, 10 times, 15 times or less, 20 times or less, 25 times or less, 30 times or less, 35 times or less, 40 times or less, 45 times or less, 50 times or less, 60 times or less, 70 times or less, 80 times or less, 90 times or less, 100 times or less, 110 times or less, 120 times or less, 130 times or less, 140 times or less, 150 times or less, 160 times or less, 170 times or less, 180 times or less, 190 times or less, 200 times or less, 210 times or less 220 times or less, 230 times or less, 240 times or less, 250 times or less, 260 times or less, 270 times or less, 280 times or less, 290 times or less, 300 times or less, 310 times or less, 320 times or less, 330 times or less, 340 times or less, 350 times or less, 360 times or less, 370 times or less, 380 times or less, 390 times or less, or 400 times or less

### Deep Bleaching

Deep Bleaching is a whitening technique conceived of and developed into a premier process by the inventor of the instant patent application. In Deep Bleaching, well-fitting and well-sealing whitening trays (e.g., extremely well-fitting and well sealing trays) are fabricated for the individual patient. The patient is instructed and provided carbamide peroxide whitening gel to wear inside the whitening trays nightly, e.g., during sleep. In some cases, the patient may be unable to or refuses to wear trays during sleep. For example, if the patient has sleep apnea and wears an oral appliance to treat the apnea, or if they have pathologic bruxism (teeth grinding) and need to wear a protective nightguard, daytime whitening may be substituted for nighttime whitening. However results may be less noticeable than with nighttime whitening. Depending on the patient's needs (e.g., the depth and quantity of stain molecules and pigment molecules), the patient is instructed to use the carbamide peroxide for nightly whitening for about 5 days, about 7 days, about 9 days, about 11 days, about 13 days, about 15 days, about 17 days, about 21 days, about 30 days, 35 days, 40 days, 45 days or less, 50 days or less, 55 days or less, 60 days or less, 65 days or less, 70 days or less, or any other length of time beneficial to the Deep Bleaching process. The patient may be instructed to continue nightly carbamide peroxide whitening for two weeks. Nightly application of carbamide peroxide generally results in active oxygenation cleansing and whitening throughout each night of the treatment period.

By the end of the patient's at-home whitening (e.g., two weeks of nightly carbamide peroxide treatments), the teeth are generally whiter and cleansed internally (e.g., the tooth microstructure may be thoroughly cleaned) due to the regular (e.g., nightly) prolonged periods of oxygenation cleansing performed throughout the night each night. However, as discussed elsewhere herein, nightly carbamide peroxide whitening suffers from low concentrations of whitening chemical break-down products. Indeed, the concentration of the whitening gel (e.g., whitening gel break-down products) is insufficient (e.g., not strong enough) to pull the stronger chromophores apart (e.g., break the chromophore bonds).

At the end of the period (e.g., two weeks) of patient conducted nighttime vital tray whitening (e.g., with carbamide peroxide), the patient may then be seen by a whitening professional for a power whitening visit. Assuming treatment protocol compliance by the patient, by this time (e.g., by the time of the power whitening office visit), the teeth may be cleansed internally (e.g., thoroughly cleansed). As such, the clean internal structure of the tooth permits comparatively high volumes of the high concentration, chemically activated hydrogen peroxide (or any other whitening chemical used in the power whitening office visit) to enter the tooth structure (e.g., rapidly and deeply penetrate the structure) in sufficient strength and volume to be able to destroy any remaining chromophore bonds (e.g., pull the strong chromophores apart).

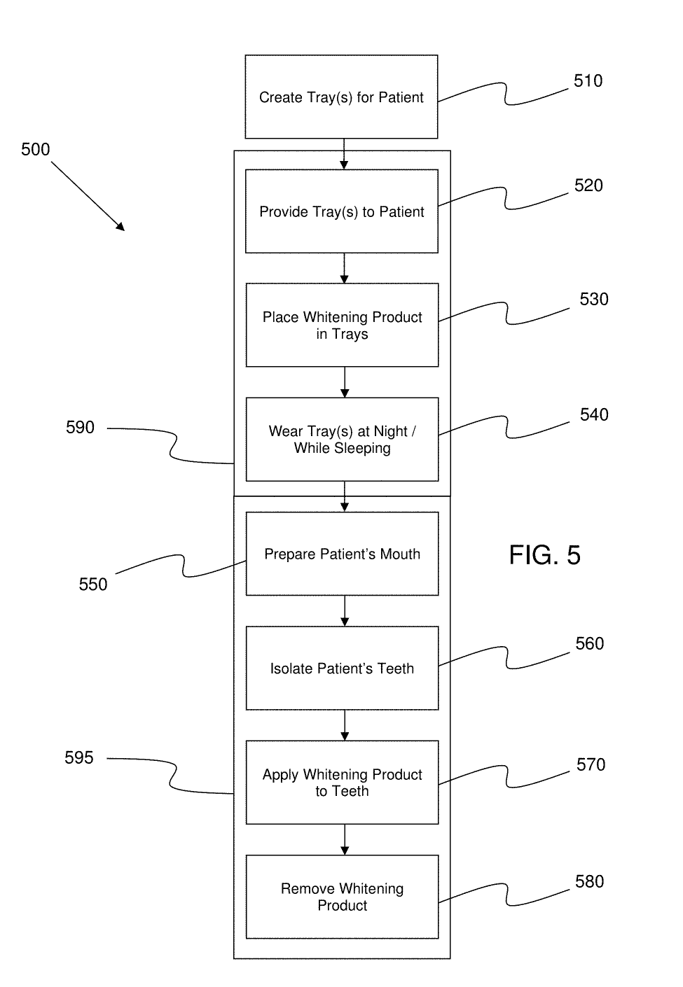

FIG. 5 shows a flow chart of one embodiment of a method for Deep Bleaching of teeth 500. The method for Deep Bleaching of teeth 500 may comprise an at home whitening portion 590 and an in-office power whitening portion 595.

At step one 510, trays may be created for the patient. The trays created for the patient may be similar if not the same as the trays created for the method for daytime vital tray whitening of teeth 100 or the method for nighttime vital tray whitening of teeth 200.

The at home whitening portion 590 of the method for combination whitening of teeth 500 comprises steps two 520 in which the trays created in step one 510 are provided to the patient, three 530 in which whitening product is placed in the trays, and four 540 in which the trays containing the whitening product are worn by the patient. The at home whitening portion 495 may be similar, if not identical to the method for nighttime vital tray whitening of teeth 200.

The in-office power whitening portion 595 comprises steps five 550 in which the patient's mouth is prepared, six 560 in which the patient's teeth are isolated, seven 570 in which whitening product is applied to the patient's teeth, and eight 580 in which the whitening product is removed from the patient's teeth. The steps of the method for combination whitening of teeth 500 in the in-office power whitening portion 595 may correspond to the steps of the method for in-office power whitening 300 discussed herein. For example: step five 550 (in which the patient's mouth is prepared) of method 500 may correspond to step one 310 (in which the patient's mouth is prepared) of method 300; step six 560 (in which the patient's teeth are isolated) of method 500 may correspond to step two 320 (in which the patient's teeth are isolated) of method 300; step seven 570 (in which the whitening product is applied to the patient's teeth) of method 500 may correspond to step three 330 (in which the whitening product is applied to the patient's teeth) of method 300; and step eight 580 (in which the whitening product is removed) of method 500 may correspond to step four 340 (in which the whitening product is removed) of method 300. Some steps in method 500 may correspond to, be similar to, or even be identical to steps in method 300. While some steps in method 500 may correspond to steps in method 300, such correspondence is not required and some steps may be partially or completely different.

The method for Deep Bleaching of teeth 500, or individual steps thereof, may be carried out until the teeth have lightened by about 5%. In some embodiments, the method for Deep Bleaching of teeth 500, or individual steps thereof, may be carried out until the teeth have lightened by about 1%, about 2%, about 3%, about 4%, about 5%, about 6%, about 7%, about 8%, about 9%, about 10%, about 12%, about 14%, about 16%, about 18%, about 20%, about 22%, about 24%, about 26%, about 28%, about 30%, about 35%, about 40%, about 45%, about 50%, about 55%, about 60%, about 65%, about 70%, or about 75%. In some embodiments, the method for Deep Bleaching of teeth 500, or individual steps thereof, may be carried out until the teeth have lightened by 1 shade, 2 shades, 3 shades, 4 shades, 5 shades, 6 shades, 7 shades, 8 shades 9 shades, 10 shades, 11 shades, 12 shades, 13 shades, 14 shades, or 15 shades, 16 shades, 17 shades, 18 shades, 19 shades, 20 shades, 25, shades, 30 shades, 35 shades, 40 shades, 45 shades, 50 shades, 55 shades, 60 shades, 65 shades, 70 shades, 75 shades, or 80 shades. In some embodiments, the desired lightening of the teeth (using either percentage or shade) may be achieved using one or more iteration of the method, or individual steps thereof. For example, the desired lightening of the teeth (using either percentage or shade) may be achieved by performing the method, or individual steps thereof, 1 time, 2 times, 3 times, 4 times, 5 times, 6 times, 7 times, 8 times 9 times, 10 times, 15 times or less, 20 times or less, 25 times or less, 30 times or less, 35 times or less, 40 times or less, 45 times or less, 50 times or less, 60 times or less, 70 times or less, 80 times or less, 90 times or less, 100 times or less, 110 times or less, 120 times or less, 130 times or less, 140 times or less, 150 times or less, 160 times or less, 170 times or less, 180 times or less, 190 times or less, 200 times or less, 210 times or less 220 times or less, 230 times or less, 240 times or less, 250 times or less, 260 times or less, 270 times or less, 280 times or less, 290 times or less, 300 times or less, 310 times or less, 320 times or less, 330 times or less, 340 times or less, 350 times or less, 360 times or less, 370 times or less, 380 times or less, 390 times or less, or 400 times or less.

### Low Concentration Whitening

The European Union Council Directive 2011/84/EU of Sep. 20, 2011 required application by EU member countries by Oct. 31, 2012 (note, the directive applies only to member countries of the European Union). The directive states that 6% is the maximum allowable percentage of hydrogen peroxide that may be used by dental professionals to whiten teeth. The directive also limits any other type of peroxide used to an equivalent strength of 6% hydrogen peroxide. Varying calculations may be used to compare equivalence between carbamide and hydrogen peroxide. However, the EU Directive rules refers to the “maximum” amount of hydrogen peroxide that could be generated by carbamide peroxide. According to the Dental Protection organization in the United Kingdom, 10% carbamide peroxide content would yield a maximum of 3.6% hydrogen peroxide. Based on this ratio, the maximum concentration of carbamide peroxide that may be used as an equivalent of 6% hydrogen peroxide is about 16.66% carbamide peroxide. Thus, products containing 16% carbamide peroxide are commonly used because it is generally accepted that they (containing 16% carbamide peroxide) release less than 6% hydrogen peroxide.

Dental offices in the European Union remain able to provide at-home nighttime and daytime vital tray whitening, however the concentration used in such treatment has been limited by the directive. Before the directive, in Europe at-home daytime tray whitening was commonly performed using about 9-12% hydrogen peroxide gel, and at-home nighttime tray whitening was performed using about 10-22% carbamide peroxide. In a similar fashion, before the directive, in Europe in-office power whitening was performed using chemically activated 25-40% hydrogen peroxide. One of the largest or most significant limitations imposed by the directive is on in-office power whitening: whereas 9-12% concentrations have been reduced only to 6% percent (or its equivalent) for at home whitening (an effective reduction of about 33-50%), concentrations of 25-40% must be reduced to the same 6% (or its equivalent) (though this in-office 6% may also be chemically activated) for in-office whitening (an effective reduction of about 76-85%). As a result of the directive, in Europe, the in-office portion of Deep Bleaching, e.g., the in-office power whitening visit after the at-home whitening, may be limited to only 6% chemically accelerated hydrogen peroxide—such concentrations are not sufficient to successfully pull apart the stronger chromophores (particularly in the limited time available in a single office visit). Indeed, some have observed a reduced interest by European dentists in offering and/or performing in-office power whitening services, likely because the results they are able to achieve when using only 6% hydrogen peroxide, e.g., chemically activated hydrogen peroxide, are frequently less apparent, e.g., not noticeable.

An alternative may be to perform in-office whitening multiple times in a comparatively short time (e.g., one, two, three, or four times a day for two days, four days, a week, two, weeks, or even more). However, such an approach would likely be inconvenient and costly for the patient. The patient would have to take off time from work or otherwise find time to go to the dental practice once or twice a day. Another requirement of the directive is that a licensed dentist is the only one that may provide in-office power whitening (e.g., no nurse, technician, hygienist, or other non-dentist professional may perform these whitening services). As a result, these visits, which must be performed personally by the dentist, would likely be costly, e.g., extremely costly, which few patients could afford.

Alternate methods of whitening that take into account the limitations imposed by the directive would be advantageous. One such method involves a partial or complete at-home nighttime whitening regime followed by a daytime whitening protocol, e.g., a daytime whitening protocol using a chemically accelerated whitening gel.

As discussed herein, at-home nighttime whitening allows cleansing of the tooth microstructure due, at least in part, to the prolonged application period and extended time of chemical activity. In some embodiments, the patient is provided the tools, products, and/or materials necessary to perform at-home nighttime whitening (e.g., whitening chemicals or gels, such as, but not limited to carbamide peroxide, and fitted whitening trays). The patient may perform (e.g., be instructed to perform) nighttime whitening each night for one or more nights. For example, between about 1 and 60 nights, between about 2 and 55 nights, between about 3 and 50 nights, between about 4 and 45 nights, between about 5 and 40 nights, between about 6 and 35 nights, between about 7 and 30 nights, between about 8 and 25 nights, between about 9 and 20 nights, between 10 and 15 nights, or any other length of time useful to attain tooth whitening as disclosed herein. These nightly whitening sessions cleanse the tooth structure through extended time periods of oxygenation.

For the reasons discussed herein, nighttime at home whitening is generally not effective at breaking down strong chromophore bonds. Therefore, at some point during or after the patient's nighttime whitening treatments (e.g., after the teeth's microstructure has been sufficiently cleansed), the patient may perform (e.g., be instructed to perform) daytime whitening, e.g., daytime whitening using a chemically accelerated whitening gel. For example, the patient may perform at-home daytime whitening concurrently with at-home nighttime whitening from the beginning of at-home whitening (e.g., at home daytime whitening is performed every day that at home nighttime whitening is performed). For example, the patient may perform at-home daytime whitening concurrently with at-home nighttime whitening after some nighttime whitening has been performed: daytime whitening may be begun after the patient has performed nighttime whitening for between about 1 and 60 nights, between about 2 and 55 nights, between about 3 and 50 nights, between about 4 and 45 nights, between about 5 and 40 nights, between about 6 and 35 nights, between about 7 and 30 nights, between about 8 and 25 nights, between about 9 and 20 nights, between 10 and 15 nights. As another example, the patient may perform at-home daytime whitening after completion of at-home nighttime whitening (e.g., after the entire nighttime whitening protocol has been finished).

The daytime whitening may be performed with a chemically accelerated 6% hydrogen peroxide. These chemical accelerators (e.g., catalysts) may result (e.g., will generally result) in a higher concentration and a higher volume of peroxide breakdown products. Because of the previous cleansing (e.g., significant cleansing) of the tooth microstructure from the well-sealed whitening trays and slow-release carbamide peroxide nighttime whitening, later applied whitening gels (e.g., daytime chemically accelerated hydrogen peroxide) may more quickly, e.g., rapidly, enter the microstructure of the teeth in greater concentrations to pull apart the stronger chromophores.

The result of nighttime whitening (e.g., using carbamide peroxide) followed by daytime whitening (e.g., using hydrogen peroxide) may be the same as if the patient first whitened at home with nighttime whitening, followed by multiple dental office visits (e.g., multiple in-office power whitening sessions). Some benefits of the method may include: more final whiteness of the teeth; convenience for the patient because the patient can wear the whitening trays at home, work, or elsewhere instead of having to present to a whitening professional (e.g., a dental practice) once or twice a day for one or two weeks; convenience for the whitening professional (e.g., dentist)—this process may take no more of the whitening professional's (e.g., dentists, or dental office's) time than when performing at-home vital tray whitening; and lower cost (e.g., far lower cost) for the patient—a small fraction of what it would cost for at-home nighttime vital tray whitening, followed by multiple visits to a whitening professional (e.g., a dental practice).

There may be various possible modifications to the method of low concentration whitening, as disclosed herein. However, one example of a low concentration whitening protocol that complies with the European directive is as follows:

### EXAMPLE I

### Low Concentration Whitening

The patient performs (e.g., is instructed to perform) at-home nighttime whitening with carbamide peroxide (with a concentration not to exceed the limits of the EU Directive). Then, the patient performs (e.g., is instructed to perform) at-home daytime whitening with chemically accelerated hydrogen peroxide (not to exceed 6% hydrogen peroxide). Some examples of various timing protocols for these at-home nighttime whitening followed by at-home daytime whitening include:
2 weeks of nighttime whitening, followed by one week of daytime whitening, once or twice a day for 60 minutes.
1-3 weeks of nighttime whitening, followed by one or more weeks of daytime whitening, 1-3 times per day for 15-60 minutes.
1-3 weeks of nighttime whitening. Daytime whitening to start 1-2 weeks after the start of nighttime whitening (in other words, there would be times where the patient is doing both nighttime whitening and daytime whitening on the same days). Daytime whitening to be done for 1 or more weeks, 1-3 times per day for 15-60 minutes.
Longer periods of either or both nighttime and daytime whitening for more difficult or resistant cases.
Shorter periods of either or both nighttime and daytime whitening when patients have unusual teeth sensitivity.
   Such methods of low concentration whitening may provide better whitening results than any system currently available, and, moreover, may result in very reasonable cost and minimal required dental office time.

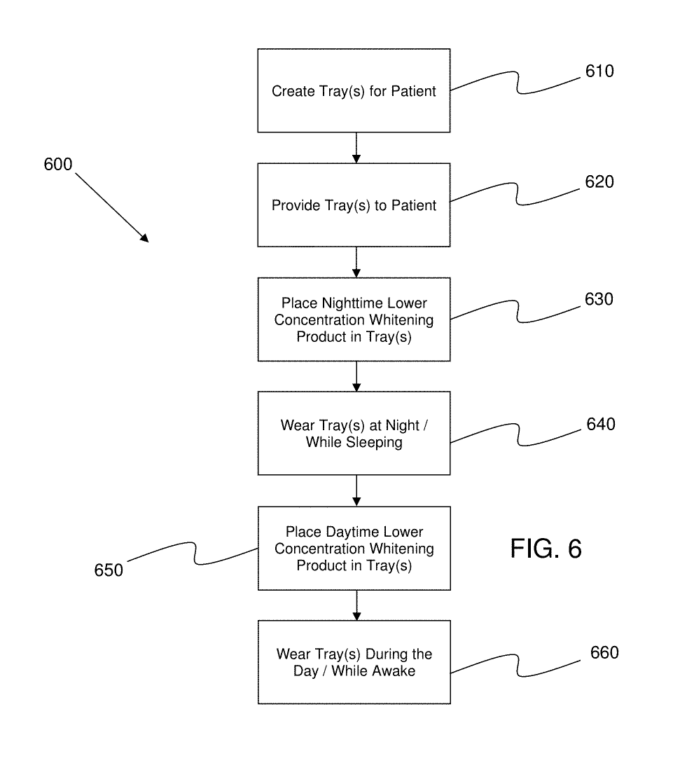

FIG. 6 shows a flow chart of one embodiment of a method for lower concentration whitening of teeth 600.

At step one 610, trays may be created for the patient. The trays created for the patient in step one 610 may be similar if not the same as the trays created for the method for daytime vital tray whitening of teeth 100 or the method for nighttime vital tray whitening of teeth 200 (or any other method disclosed herein). At step two 620, the tray created in step one 610, including one or more of a maxillary tray and a mandibular tray, are provided to the patient.

The patient may be instructed regarding one or more steps of the method for lower concentration whitening of teeth 600. Furthermore, the patient may be provided one or more products, e.g., whitening products, in addition to the trays.

At step three 630, the nighttime lower concentration whitening product, e.g., a lower concentration whitening product compliant with the EU directive discussed herein, is placed in the tray. The nighttime whitening product may be placed in the tray by the patient, a member of the patient's family, a caretaker, or a dental professional, or any other person capable of placing the whitening product in the tray. In some embodiments, the nighttime whitening product is a whitening gel. In some embodiments, the nighttime whitening product is a carbamide peroxide whitening gel having a concentration of less than about 16.66, less than about 16%, or less than about 15%. In some embodiments, the nighttime whitening product is a carbamide peroxide whitening gel having any concentration, e.g., a concentration, compliant with the EU directive. In some embodiments, the nighttime whitening product is a chemically accelerated carbamide peroxide whitening gel. In some embodiments, the nighttime lower concentration whitening product is a carbamide peroxide whitening gel having a concentration as high as possible without violating the EU directive. In some embodiments, the nighttime whitening product is a whitening gel comprising a chemical that breaks down slowly into whitening products. In some embodiments, the nighttime whitening product is a whitening gel comprising a chemical that breaks down slowly enough so as to remain active (e.g., maintain whitening activity) for at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, or at least about 24 hours. In some embodiments, the nighttime whitening product is a whitening gel comprising a chemical that breaks down slowly enough so as to remain active (e.g., maintain whitening activity) for between about 2-24 hours, between about 3-22 hours, between about 4-20 hours, between about 5-18 hours, between about 6-16 hours, between about 7-14 hours, between about 8-12 hours, or between about 9-11 hours.

At step four 640, the tray containing the nighttime whitening product is placed in the mouth and worn for a time period during the nighttime. The nighttime whitening product may be placed in the tray and then in tray placed in the mouth shortly before, e.g., immediately before, the patient goes to bed. In some embodiments, the tray containing the whitening product is worn for the entire time the patient is asleep, e.g., about 6-8 hours. In some embodiments, the tray containing the nighttime whitening product is worn for less than about 12 hours, less than about 11.5 hours, less than about 11 hours, less than about 10.5 hours, less than about 10 hours, less than about 9.5 hours, less than about 9 hours, less than about 8.75 hours, less than about 8.5 hours, less than about 8.25 hours, less than about 8 hours, less than about less than about 7.75 hours, less than about 7.5 hours, less than about 7.25 hours, less than about 7 hours, less than about 6.75 hours, less than about 6.5 hours, less than about 6.25 hours, less than about 6 hours, less than about 5.75 hours, less than about 5.5 hours, less than about 5.25 hours, less than about 5 hours, less than about 4.75 hours, less than about 4.5 hours, less than about 4.25 hours, less than about 4 hours, less than about 3.75 hours, less than about 3.5 hours, less than about 3.25 hours, or less than about 3 hours. In some embodiments, the tray containing the nighttime whitening product is worn for between about 2-20 hours, between about 2.25-19.5 hours, between about 2.5-19 hours, between about 2.75-18.5 hours, between about 3-18 hours, between about 3.25-17.5 hours, between about 3.5-17 hours, between about 3.75-16.5 hours, between about 4-16 hours, between about 4.25-15.5 hours, between about 4.5-15 hours, between about 4.75-14.5 hours, between about 5-14 hours, between about 5.25-13.5 hours, between about 5.5-13 hours, between about 5.75-12.5 hours, between about 6-11.5 hours, between about 6.25-11 hours, between about 6.5-10.5 hours, between about 6.75-10 hours, between about 7-9.5 hours, between about 7.25-9 hours, between about 7.5-8.5 hours, or between about 7.75-8 hours, or any other length of time that is beneficial to the desired level or degree of whitening.

Steps three 630 and four 640 of the method for lower concentration whitening of teeth 600 may be carried out for any number of nights, e.g., consecutive nights, or non-consecutive nights over a time period. For example, steps three 630 and four 640 of the method for lower concentration whitening of teeth 600 may be carried out for 1 night, 2, nights, 3 nights, 4 nights, 5 nights, 6 nights, 7 nights, 8 nights, 9 nights, 10 nights, 11 nights, 12 nights, 13 nights, 14 nights, 15 nights, 16 nights, 17 nights, 18 nights, 19 nights, 20 nights, 21 nights, 22 nights, 24 nights, 26 nights, 28 nights, 30 nights, 32 nights, 34 nights, 36 nights, 38 nights, 40 nights, 45 nights, 50 nights, 55 nights, 60 nights, 65 nights, 70 nights, 75 nights, 80 nights, 85 nights, 90 nights 95 nights, 100 nights, 105 nights, 110 nights, 115 nights, 120 nights, 125 nights, 130 nights, 135 nights, 140 nights, 145 nights, 150 nights, 155 nights, 160 nights, 170 nights, 180 nights, 190 nights, 200 nights, 210 nights, 220 nights, 230 nights, 240 nights, 250 nights, 260 nights, 270 nights, 280 nights, 290 nights, 300 nights, 310 nights, 320 nights, 330 nights, 340 nights, 350 nights, 360 nights, or more nights. In some embodiments, steps three 630 and four 640 of the method for lower concentration whitening of teeth 600 are carried out for more than 40 nights. In some embodiments, steps three 630 and four 640 of the method for lower concentration whitening of teeth 600 are carried out for between about 1 and 60 nights, between about 2 and 55 nights, between about 3 and 50 nights, between about 4 and 45 nights, between about 5 and 40 nights, between about 6 and 35 nights, between about 7 and 30 nights, between about 8 and 25 nights, between about 9 and 20 nights, between 10 and 15 nights, or any other length of time useful to attain tooth whitening as disclosed herein. In some embodiments, the steps three 630 and four 640 of the method for lower concentration whitening of teeth 600 are carried out until the teeth reach a desired shade, e.g., lighten to a desired shade.

At step five 650, the daytime lower concentration whitening product is placed in the tray. The daytime lower concentration whitening product may be placed in the tray by the patient, a member of the patient's family, a caretaker, or a dental professional, or any other person capable of placing the whitening product in the tray. In some embodiments, the daytime lower concentration whitening product is a whitening gel. In some embodiments, the daytime lower concentration whitening product is a hydrogen peroxide whitening gel. In some embodiments, the daytime lower concentration whitening product is a hydrogen peroxide whitening gel having a concentration of less than or about 6%. In some embodiments, the daytime lower concentration whitening product is a chemically accelerated hydrogen peroxide whitening gel having a concentration of less than or about 6%. In some embodiments, the daytime lower concentration whitening product is a hydrogen peroxide whitening gel having any concentration, e.g., a concentration, compliant with the EU directive. In some embodiments, the daytime lower concentration whitening product is a hydrogen peroxide whitening gel having a concentration as high as possible without violating the EU directive. In some embodiments, the daytime lower concentration whitening product is a chemically accelerated hydrogen peroxide whitening gel. In some embodiments, the whitening product is a whitening gel comprising a chemical that breaks down quickly into whitening products.

At step six 660, the tray containing the daytime lower concentration whitening product is placed in the mouth and worn for a time period during the daytime. In some embodiments, the tray containing the whitening product is worn for less than about 180 minutes, less than about 170 minutes, less than about 160 minutes, less than about 150 minutes, less than about 140 minutes, less than about 130 minutes, less than about 120 minutes, less than about 110 minutes, less than about 100 minutes, less than about 90 minutes, less than about 80 minutes, less than about 70 minutes, less than about 60 minutes, less than about 50 minutes, less than about 40 minutes, less than about 30 minutes, less than about 20 minutes, less than about 10 minutes, or less than about 5 minutes. In some embodiments, the tray containing the whitening product is worn for between about 5-300 minutes, about 10-240 minutes, about 15-180 minutes, about 20-120 minutes, about 25-60 minutes, about 30-45 minutes, or any other length of time that is beneficial to the desired level or degree of whitening.

In some embodiments of the method for lower concentration whitening of teeth 600, steps five 650 and six 660 may be performed more than once (e.g., repeated) during a single day. In some embodiments, in a single day steps five 650 and six 660 are performed 2 times, 3 times, 4 times, 5 times, 6 times, 7 times, 8 times, 9 times, or 10 times.

Steps five 650 and six 660 of the method for lower concentration whitening of teeth 600 may be carried out for any number of days, e.g., consecutive days, or non-consecutive days over a time period. For example, steps five 650 and six 660 of the method for lower concentration whitening of teeth 600 may be carried out for 1 day, 2, days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 24 days, 26 days, 28 days, 30 days, 32 days, 34 days, 36 days, 38 days, 40 days, 45 days, 50 days, 55 days, 60 days, 65 days, 70 days, 75 days, 80 days, 85 days, 90 days, 95 days, 100 days, 105 days, 110 days, 115 days, 120 days, 125 days, 130 days, 135 days, 140 days, 145 days, 150 days, 155 days, 160 days, 170 days, 180 days, 190 days, 200 days, 210 days, 220 days, 230 days, 240 days, 250 days, 260 days, 270 days, 280 days, 290 days, 300 days, 310 days, 320 days, 330 days, 340 days, 350 days, 360 days, or more days. In some embodiments, steps five 650 and six 660 of the method for lower concentration whitening of teeth 600 is carried out for more than 40 days. In some embodiments, steps five 650 and six 660 of the method for lower concentration whitening of teeth 600 is carried out until the teeth reach a desired shade, e.g., lighten to a desired shade.

In some embodiments, the patient may perform the nighttime protocol of steps three 630 and four 640 concurrently with daytime protocol of steps five 650 and six 660, e.g., the patient may wear the trays containing the daytime whitening product during the day after they wore the trays containing the nighttime whitening product. For example, daytime whitening including steps five 650 and six 660 may be begun after the patient has performed nighttime whitening including steps three 630 and four 640 for between about 1 and 60 nights, between about 2 and 55 nights, between about 3 and 50 nights, between about 4 and 45 nights, between about 5 and 40 nights, between about 6 and 35 nights, between about 7 and 30 nights, between about 8 and 25 nights, between about 9 and 20 nights, between 10 and 15 nights. In some embodiments, the patient does not perform daytime protocol of steps five 650 and six 660 until they have finished the nighttime protocol of steps three 630 and four 640.

The method for lower concentration whitening of teeth 600, or individual steps thereof, may be carried out until the teeth have lightened by about 5%. In some embodiments, the method for lower concentration whitening of teeth 600, or individual steps thereof, may be carried out until the teeth have lightened by about 1%, about 2%, about 3%, about 4%, about 5%, about 6%, about 7%, about 8%, about 9%, about 10%, about 12%, about 14%, about 16%, about 18%, about 20%, about 22%, about 24%, about 26%, about 28%, about 30%, about 35%, about 40%, about 45%, about 50%, about 55%, about 60%, about 65%, about 70%, or about 75%. In some embodiments, the method for lower concentration whitening of teeth 600, or individual steps thereof, may be carried out until the teeth have lightened by 1 shade, 2 shades, 3 shades, 4 shades, 5 shades, 6 shades, 7 shades, 8 shades 9 shades, 10 shades, 11 shades, 12 shades, 13 shades, 14 shades, or 15 shades, 16 shades, 17 shades, 18 shades, 19 shades, 20 shades, 25, shades, 30 shades, 35 shades, 40 shades, 45 shades, 50 shades, 55 shades, 60 shades, 65 shades, 70 shades, 75 shades, or 80 shades. In some embodiments, the desired lightening of the teeth (using either percentage or shade) may be achieved using one or more iteration of the method, or individual steps thereof. For example, the desired lightening of the teeth (using either percentage or shade) may be achieved by performing the method, or individual steps thereof, 1 time, 2 times, 3 times, 4 times, 5 times, 6 times, 7 times, 8 times 9 times, 10 times, 15 times or less, 20 times or less, 25 times or less, 30 times or less, 35 times or less, 40 times or less, 45 times or less, 50 times or less, 60 times or less, 70 times or less, 80 times or less, 90 times or less, 100 times or less, 110 times or less, 120 times or less, 130 times or less, 140 times or less, 150 times or less, 160 times or less, 170 times or less, 180 times or less, 190 times or less, 200 times or less, 210 times or less 220 times or less, 230 times or less, 240 times or less, 250 times or less, 260 times or less, 270 times or less, 280 times or less, 290 times or less, 300 times or less, 310 times or less, 320 times or less, 330 times or less, 340 times or less, 350 times or less, 360 times or less, 370 times or less, 380 times or less, 390 times or less, or 400 times or less.

### Alternative Nighttime/Daytime Combination Whitening

Some whitening professionals may not be limited in the concentrations of whitening gels they may use. In these cases, higher concentrations of whitening gels may be advantageously used to reduce the frequency and/or time of whitening treatments and/or improve the whitening treatment outcome (e.g., achieve better whitening). In such cases, whitening methods may involve a partial or complete at-home higher-strength (e.g., concentration) nighttime whitening regime followed by an at-home higher-strength daytime whitening protocol. One or more steps of the method/process may be performed in-office (particularly steps relying on or using particularly high concentrations of whitening gels).

As discussed above, at-home nighttime whitening allows cleansing of the tooth microstructure due, at least in part, to the prolonged application period and extended time of chemical activity. In some embodiments, the patient is provided the tools, products, and/or materials necessary to perform at-home nighttime whitening (e.g., whitening chemicals or gels, such as, but not limited to carbamide peroxide, and fitted whitening trays). The patient may perform (e.g., be instructed to perform) nighttime whitening each night for one or more nights. For example, between about 1 and 60 nights, between about 2 and 55 nights, between about 3 and 50 nights, between about 4 and 45 nights, between about 5 and 40 nights, between about 6 and 35 nights, between about 7 and 30 nights, between about 8 and 25 nights, between about 9 and 20 nights, between 10 and 15 nights, or any other length of time useful to attain tooth whitening as disclosed herein. These nightly whitening sessions cleanse the tooth structure through extended time periods of oxygenation. The nighttime whitening gel used may be a carbamide peroxide gel and may have a concentration of between about 1-50%, between about 2-47%, between about 3-45%, between about 3-43%, between about 4-40%, between about 5-37%, between about 6-35%, between about 7-33%, between about 8-30%, between about 9-27%, between about 10-25%, or any other concentration that advantageously whitens the teeth according to any of the methods disclosed herein. The whitening gel used may be a carbamide peroxide gel mixed with any other whitening gel, such as, but not limited to a hydrogen peroxide gel.

For the reasons discussed elsewhere herein, nighttime at home whitening may be less effective at breaking down strong chromophore bonds. Therefore, at some point during or after the patient's nighttime whitening treatments (e.g., after the teeth's microstructure has been sufficiently cleansed), the patient may perform (e.g., be instructed to perform) daytime whitening. For example, the patient may perform at-home daytime whitening concurrently with at-home nighttime whitening from the beginning of at-home whitening (e.g., at home daytime whitening is performed every day that at home nighttime whitening is performed). For example, the patient may perform at-home daytime whitening concurrently with at-home nighttime whitening after some nighttime whitening has been performed: daytime whitening may be begun after the patient has performed nighttime whitening for between about 1 and 60 nights, between about 2 and 55 nights, between about 3 and 50 nights, between about 4 and 45 nights, between about 5 and 40 nights, between about 6 and 35 nights, between about 7 and 30 nights, between about 8 and 25 nights, between about 9 and 20 nights, between 10 and 15 nights. As another example, the patient may perform at-home daytime whitening after completion of at-home nighttime whitening (e.g., after the entire nighttime whitening protocol has been finished).

The daytime whitening gel used may be hydrogen peroxide and may have a concentration of between about 1-25%, between about 3-23%, between about 5-20%, between about 7-17%, between about 10-15%, or any other concentration that advantageously whitens the teeth according to any of the methods disclosed herein. The daytime whitening gel may be hydrogen peroxide mixed with any other type of whitening gel or chemical, such as, but not limited to carbamide peroxide. The daytime whitening gel may be any whitening gel/chemical (e.g., including any quick break-down whitening gel/chemical). The daytime whitening gel/chemical may be a mixture of hydrogen peroxide gel (e.g., a hydrogen peroxide gel having a concentration as disclosed herein) mixed with a carbamide peroxide gel (e.g., a carbamide peroxide gel having a concentration as disclosed herein) in a hydrogen peroxide gel to carbamide peroxide gel ratio of about 99/1, about 95/5, about 90/10, about 85/15, about 80/20, about 75/25, about 70/30, about 65/35, about 60/40, about 55/45, about 50/50 or any other ratio that advantageously whitens the teeth according to any of the methods disclosed herein. Because of the previous cleansing (e.g., significant cleansing) of the tooth microstructure from the well-sealed whitening trays and slow-release carbamide peroxide nighttime whitening, later applied whitening gels (e.g., daytime hydrogen peroxide) may rapidly enter the microstructure of the teeth in greater concentrations to pull apart the stronger chromophores.

The result of nighttime whitening (e.g., using carbamide peroxide) followed by daytime whitening (e.g., using hydrogen peroxide or other rapid-release whitening gel or chemical) may be the same as if the patient first whitened at home with nighttime whitening, followed by multiple dental office visits (e.g., multiple in-office power whitening sessions). Some benefits of the method may include: more final whiteness of the teeth; convenience for the patient because the patient can wear the whitening trays at home, work, or elsewhere instead of having to present to a whitening professional (e.g., a dental practice) once or twice a day for one or two weeks; convenience for the whitening professional (e.g., dentist)—this process may take no more of the whitening professional's (e.g., dentists, or dental office's) time than when performing at-home vital tray whitening; and lower cost (e.g., far lower cost) for the patient—a small fraction of what it would cost for at-home nighttime vital tray whitening, followed by multiple visits (or even just a single visit) to a whitening professional (e.g., a dental practice).

There may be various possible modifications to the method of low concentration whitening, as disclosed herein. However, one example of a whitening protocol is as follows:

### EXAMPLE II

### Alternative Nighttime/Daytime Combination Whitening

The patient performs (e.g., is instructed to perform) at-home nighttime whitening with a whitening gel made up entirely of a 35% carbamide peroxide gel (e.g., unaccelerated carbamide peroxide (e.g., carbamide peroxide that has not been accelerated)), a 10-22% carbamide peroxide gel, or a 15-16% carbamide peroxide gel. Then, the patient performs (e.g., is instructed to perform) at-home daytime whitening with a whitening gel made of about 75% of a 12-16% hydrogen peroxide gel (e.g., unaccelerated hydrogen peroxide at a concentration of about 10%, 11%, 12%, 13%, 14%, 15%, 16%, 17%, or 18%) and about 25% of a 35-38% carbamide peroxide gel (e.g., unaccelerated carbamide peroxide at a concentration of about 30%, 31%, 32%, 33%, 34%, 35%, 36%, 37%, 38%, 39%, or 40%). As discussed herein, any portion of the method may be performed in-office (e.g., the daytime whitening may be performed in-office). Some examples of various timing protocols for these at-home nighttime whitening followed by at-home daytime whitening include:
2 weeks of nighttime whitening, followed by one week of daytime whitening, once or twice a day for 60 minutes.
1-3 weeks of nighttime whitening, followed by one or more weeks of daytime whitening, 1-3 times per day for 15-60 minutes.
1-3 weeks of nighttime whitening. Daytime whitening to start 1-2 weeks after the start of nighttime whitening (in other words, there would be times where the patient is doing both nighttime whitening and daytime whitening on the same days). Daytime whitening to be done for 1 or more weeks, 1-3 times per day for 15-60 minutes.
Longer periods of either or both nighttime and daytime whitening for more difficult or resistant cases.
Shorter periods of either or both nighttime and daytime whitening when patients have unusual teeth sensitivity.
   Such methods of low concentration whitening may provide better whitening results than any system currently available, and, moreover, may result in very reasonable cost and minimal required dental office time.

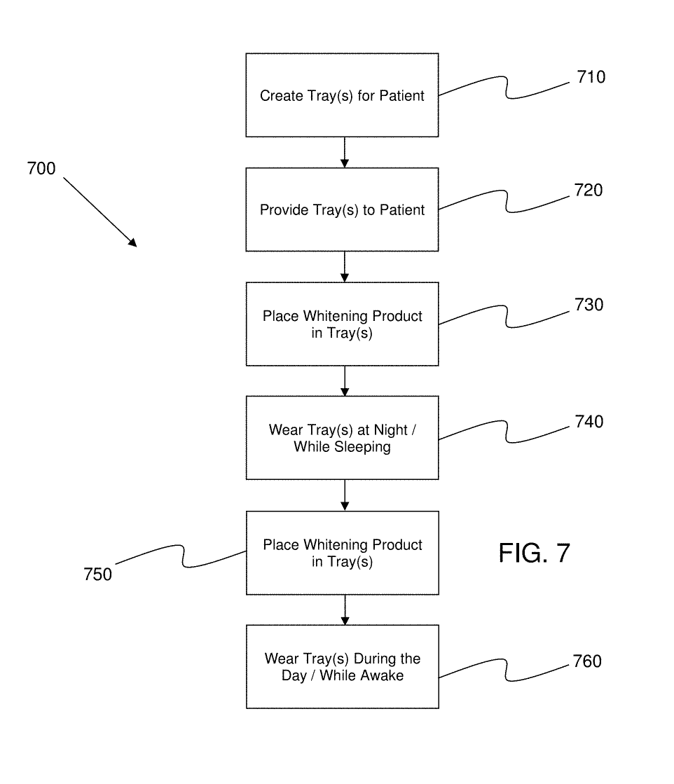

FIG. 7 shows a flow chart of one embodiment of a method for alternative combination whitening of teeth 700.

At step one 710, trays may be created for the patient. The trays created for the patient in step one 710 may be similar if not the same as the trays created for the method for daytime vital tray whitening of teeth 100 or the method for nighttime vital tray whitening of teeth 200 (or any other method disclosed herein). At step two 720, the tray created in step one 710, including one or more of a maxillary tray and a mandibular tray, are provided to the patient.

The patient may be instructed regarding one or more steps of the method for alternative combination whitening of teeth 700. Furthermore, the patient may be provided one or more products, e.g., whitening products, in addition to the trays.

At step three 730, the whitening product is placed in the tray. The whitening product may be placed in the tray by the patient, a member of the patient's family, a caretaker, or a dental professional, or any other person capable of placing the whitening product in the tray. In some embodiments, the whitening product is a whitening gel. In some embodiments, the whitening product is a carbamide peroxide whitening gel. In some embodiments, the whitening product is a carbamide peroxide whitening gel having a concentration of less than about 70%., less than about 65%, less than about 60%, less than about 55%, less than about 50%, less than about 45%, less than about 40%, less than about 35%, less than about 30%, less than about 25%, less than about 20%, less than about 17.5%, less than about 15%, less than about 12.5%, less than about 10%, less than about 9%, less than about 8%, less than about 7%, less than about 6%, less than about 5%, less than about 4%, less than about 3%, or less than about 2%. In some embodiments, the whitening product is a carbamide peroxide whitening gel having a concentration of between about 7-50%, between about 8-45%, between about 9-40%, between about 10-35%, between about 11-30%, between about 12-25%, between about 13-20%, or between about 14-17%. In some embodiments, the whitening product is a carbamide peroxide whitening gel having a concentration of between about 10-70%, between about 11-65%, between about 12-60%, between about 13-55%, between about 14-50%, between about 15-45%, between about 16-40%, between about 17-35%, between about 18-30%, or between about 19-25%. In some embodiments, the whitening product is a carbamide peroxide gel and may have a concentration of between about 1-50%, between about 2-47%, between about 3-45%, between about 3-43%, between about 4-40%, between about 5-37%, between about 6-35%, between about 7-33%, between about 8-30%, between about 9-27%, between about 10-25%, or any other concentration that advantageously whitens the teeth according to any of the methods disclosed herein. The whitening gel used may be a carbamide peroxide gel mixed with any other whitening gel, such as, but not limited to a hydrogen peroxide gel. In some embodiments, the whitening product is a chemically accelerated carbamide peroxide whitening gel. In some embodiments, the whitening product is a carbamide peroxide whitening gel having a concentration as high as possible without damaging the oral structures or mucosa or causing pain to the patient. In some embodiments, the whitening product is a whitening gel comprising a chemical that breaks down slowly into whitening products. In some embodiments, the whitening product is a whitening gel comprising a chemical that breaks down slowly enough so as to remain active (e.g., maintain whitening activity) for at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, or at least about 24 hours. In some embodiments, the whitening product is a whitening gel comprising a chemical that breaks down slowly enough so as to remain active (e.g., maintain whitening activity) for between about 2-24 hours, between about 3-22 hours, between about 4-20 hours, between about 5-18 hours, between about 6-16 hours, between about 7-14 hours, between about 8-12 hours, or between about 9-11 hours.

At step four 740, the tray containing the nighttime whitening product is placed in the mouth and worn for a time period during the nighttime. The nighttime whitening product may be placed in the tray and then in tray placed in the mouth shortly before, e.g., immediately before, the patient goes to bed. In some embodiments, the tray containing the whitening product is worn for the entire time the patient is asleep, e.g., about 6-8 hours. In some embodiments, the tray containing the nighttime whitening product is worn for less than about 12 hours, less than about 11.5 hours, less than about 11 hours, less than about 10.5 hours, less than about 10 hours, less than about 9.5 hours, less than about 9 hours, less than about 8.75 hours, less than about 8.5 hours, less than about 8.25 hours, less than about 8 hours, less than about less than about 7.75 hours, less than about 7.5 hours, less than about 7.25 hours, less than about 7 hours, less than about 6.75 hours, less than about 6.5 hours, less than about 6.25 hours, less than about 6 hours, less than about 5.75 hours, less than about 5.5 hours, less than about 5.25 hours, less than about 5 hours, less than about 4.75 hours, less than about 4.5 hours, less than about 4.25 hours, less than about 4 hours, less than about 3.75 hours, less than about 3.5 hours, less than about 3.25 hours, or less than about 3 hours. In some embodiments, the tray containing the nighttime whitening product is worn for between about 2-20 hours, between about 2.25-19.5 hours, between about 2.5-19 hours, between about 2.75-18.5 hours, between about 3-18 hours, between about 3.25-17.5 hours, between about 3.5-17 hours, between about 3.75-16.5 hours, between about 4-16 hours, between about 4.25-15.5 hours, between about 4.5-15 hours, between about 4.75-14.5 hours, between about 5-14 hours, between about 5.25-13.5 hours, between about 5.5-13 hours, between about 5.75-12.5 hours, between about 6-11.5 hours, between about 6.25-11 hours, between about 6.5-10.5 hours, between about 6.75-10 hours, between about 7-9.5 hours, between about 7.25-9 hours, between about 7.5-8.5 hours, or between about 7.75-8 hours, or any other length of time that is beneficial to the desired level or degree of whitening.

Steps three 730 and four 740 of the method for alternative combination whitening of teeth 700 may be carried out for any number of nights, e.g., consecutive nights, or non-consecutive nights over a time period. For example, steps three 730 and four 740 of the method for alternative combination whitening of teeth 700 may be carried out for 1 night, 2, nights, 3 nights, 4 nights, 5 nights, 6 nights, 7 nights, 8 nights, 9 nights, 10 nights, 11 nights, 12 nights, 13 nights, 14 nights, 15 nights, 16 nights, 17 nights, 18 nights, 19 nights, 20 nights, 21 nights, 22 nights, 24 nights, 26 nights, 28 nights, 30 nights, 32 nights, 34 nights, 36 nights, 38 nights, 40 nights, 45 nights, 50 nights, 55 nights, 60 nights, 65 nights, 70 nights, 75 nights, 80 nights, 85 nights, 90 nights 95 nights, 100 nights, 105 nights, 110 nights, 115 nights, 120 nights, 125 nights, 130 nights, 135 nights, 140 nights, 145 nights, 150 nights, 155 nights, 160 nights, 170 nights, 180 nights, 190 nights, 200 nights, 210 nights, 220 nights, 230 nights, 240 nights, 250 nights, 260 nights, 270 nights, 280 nights, 290 nights, 300 nights, 310 nights, 320 nights, 330 nights, 340 nights, 350 nights, 360 nights, or more nights. In some embodiments, steps three 730 and four 740 of the method for alternative combination whitening of teeth 700 are carried out for more than 40 nights. In some embodiments, steps three 730 and four 740 of the method for alternative combination whitening of teeth 700 are carried out for between about 1 and 60 nights, between about 2 and 55 nights, between about 3 and 50 nights, between about 4 and 45 nights, between about 5 and 40 nights, between about 6 and 35 nights, between about 7 and 30 nights, between about 8 and 25 nights, between about 9 and 20 nights, between 10 and 15 nights, or any other length of time useful to attain tooth whitening as disclosed herein. In some embodiments, steps three 730 and four 740 of the method for alternative combination whitening of teeth 700 are carried out until the teeth reach a desired shade, e.g., lighten to a desired shade.

At step five 750, the whitening product is placed in the tray. The whitening product may be placed in the tray by the patient, a member of the patient's family, a caretaker, or a dental professional, or any other person capable of placing the whitening product in the tray. In some embodiments, the whitening product is a whitening gel. In some embodiments, the whitening product is a hydrogen peroxide whitening gel. In some embodiments, the whitening product is a hydrogen peroxide whitening gel having a concentration of less than about 50%, less than about 45%, less than about 40%, less than about 35%, less than about 30%, less than about 25%, less than about 20%, less than about 17.5%, less than about 15%, less than about 12.5%, less than about 10%, less than about 9%, less than about 8%, less than about 7%, less than about 6%, less than about 5%, less than about 4%, less than about 3%, or less than about 2%. In some embodiments, the whitening product is a hydrogen peroxide whitening gel having a concentration of between about 7-50%, between about 8-45%, between about 9-40%, between about 10-35%, between about 11-30%, between about 12-25%, between about 13-20%, or between about 14-17%. In some embodiments, the whitening product is a hydrogen peroxide whitening gel having a concentration of between about 1-25%, between about 3-23%, between about 5-20%, between about 7-17%, or between about 10-15%. In some embodiments, the whitening product is a chemically accelerated hydrogen peroxide whitening gel. In some embodiments, the whitening product is a hydrogen peroxide whitening gel having a concentration as high as possible without damaging the oral structures or mucosa or causing pain to the patient. In some embodiments, the whitening product is a whitening gel comprising a chemical that breaks down quickly into whitening products. In some embodiments, the whitening product is a mixture of hydrogen peroxide gel (e.g., a hydrogen peroxide gel having a concentration as disclosed herein) mixed with a carbamide peroxide gel (e.g., a carbamide peroxide gel having a concentration as disclosed herein) in a hydrogen peroxide gel to carbamide peroxide gel ratio of about 99/1, about 98/2, about 96/4, about 94/6, about 92/8, about 90/10, about 88/12, about 86/14, about 84/16, about 82/18, about 80/20, about 78/22, about 76/24, about 74/26, about 72/28, about 70/30, about 68/32, about 66/34, about 64/36, about 62/38, about 60/40, about 58/42, about 56/44, about 54/46, about 52/48, about 50/50 or any other ratio that advantageously whitens the teeth according to any of the methods disclosed herein.

At step six 760, the tray containing the whitening product is placed in the mouth and worn for a time period during the daytime, e.g., while awake. In some embodiments, the tray containing the whitening product is worn for less than about 180 minutes, less than about 170 minutes, less than about 160 minutes, less than about 150 minutes, less than about 140 minutes, less than about 130 minutes, less than about 120 minutes, less than about 110 minutes, less than about 100 minutes, less than about 90 minutes, less than about 80 minutes, less than about 70 minutes, less than about 60 minutes, less than about 50 minutes, less than about 40 minutes, less than about 30 minutes, less than about 20 minutes, less than about 10 minutes, or less than about 5 minutes. In some embodiments, the tray containing the whitening product is worn for between about 5-300 minutes, about 10-240 minutes, about 15-180 minutes, about 20-120 minutes, about 25-60 minutes, about 30-45 minutes, or any other length of time that is beneficial to the desired level or degree of whitening.

In some embodiments of the method for alternative combination whitening of teeth 700, steps five 750 and six 760 may be performed more than once (e.g., repeated) during a single day. In some embodiments, in a single day steps five 750 and six 760 are performed 2 times, 3 times, 4 times, 5 times, 6 times, 7 times, 8 times, 9 times, or 10 times.

Steps five 750 and six 760 of the method for alternative combination whitening of teeth 700 may be carried out for any number of days, e.g., consecutive days, or non-consecutive days over a time period. For example, steps five 750 and six 760 of the method for alternative combination whitening of teeth 700 may be carried out for 1 day, 2, days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 24 days, 26 days, 28 days, 30 days, 32 days, 34 days, 36 days, 38 days, 40 days, 45 days, 50 days, 55 days, 60 days, 65 days, 70 days, 75 days, 80 days, 85 days, 90 days, 95 days, 100 days, 105 days, 110 days, 115 days, 120 days, 125 days, 130 days, 135 days, 140 days, 145 days, 150 days, 155 days, 160 days, 170 days, 180 days, 190 days, 200 days, 210 days, 220 days, 230 days, 240 days, 250 days, 260 days, 270 days, 280 days, 290 days, 300 days, 310 days, 320 days, 330 days, 340 days, 350 days, 360 days, or more days. In some embodiments, steps five 750 and six 760 of the method for alternative combination whitening of teeth 700 are carried out for more than 40 days. In some embodiments, steps five 750 and six 760 of the method for alternative combination whitening of teeth 700 are carried out until the teeth reach a desired shade, e.g., lighten to a desired shade.

In some embodiments, the patient may perform the nighttime protocol of steps three 730 and four 740 concurrently with daytime protocol of steps five 750 and six 760, e.g., the patient may wear the trays containing the daytime whitening product during the day after they wore the trays containing the nighttime whitening product. For example, daytime whitening including steps five 750 and six 760 may be begun after the patient has performed nighttime whitening including steps three 730 and four 740 for between about 1 and 60 nights, between about 2 and 55 nights, between about 3 and 50 nights, between about 4 and 45 nights, between about 5 and 40 nights, between about 6 and 35 nights, between about 7 and 30 nights, between about 8 and 25 nights, between about 9 and 20 nights, between 10 and 15 nights. In some embodiments, the patient does not perform daytime protocol of steps five 750 and six 760 until they have finished the nighttime protocol of steps three 730 and four 740.

The method for alternative combination whitening of teeth 700, or individual steps thereof, may be carried out until the teeth have lightened by about 5%. In some embodiments, the method for alternative combination whitening of teeth 700, or individual steps thereof, may be carried out until the teeth have lightened by about 1%, about 2%, about 3%, about 4%, about 5%, about 6%, about 7%, about 8%, about 9%, about 10%, about 12%, about 14%, about 16%, about 18%, about 20%, about 22%, about 24%, about 26%, about 28%, about 30%, about 35%, about 40%, about 45%, about 50%, about 55%, about 60%, about 65%, about 70%, or about 75%. In some embodiments, the method for alternative combination whitening of teeth 700, or individual steps thereof, may be carried out until the teeth have lightened by 1 shade, 2 shades, 3 shades, 4 shades, 5 shades, 6 shades, 7 shades, 8 shades 9 shades, 10 shades, 11 shades, 12 shades, 13 shades, 14 shades, or 15 shades, 16 shades, 17 shades, 18 shades, 19 shades, 20 shades, 25, shades, 30 shades, 35 shades, 40 shades, 45 shades, 50 shades, 55 shades, 60 shades, 65 shades, 70 shades, 75 shades, or 80 shades. In some embodiments, the desired lightening of the teeth (using either percentage or shade) may be achieved using one or more iteration of the method, or individual steps thereof. For example, the desired lightening of the teeth (using either percentage or shade) may be achieved by performing the method, or individual steps thereof, 1 time, 2 times, 3 times, 4 times, 5 times, 6 times, 7 times, 8 times 9 times, 10 times, 15 times or less, 20 times or less, 25 times or less, 30 times or less, 35 times or less, 40 times or less, 45 times or less, 50 times or less, 60 times or less, 70 times or less, 80 times or less, 90 times or less, 100 times or less, 110 times or less, 120 times or less, 130 times or less, 140 times or less, 150 times or less, 160 times or less, 170 times or less, 180 times or less, 190 times or less, 200 times or less, 210 times or less 220 times or less, 230 times or less, 240 times or less, 250 times or less, 260 times or less, 270 times or less, 280 times or less, 290 times or less, 300 times or less, 310 times or less, 320 times or less, 330 times or less, 340 times or less, 350 times or less, 360 times or less, 370 times or less, 380 times or less, 390 times or less, or 400 times or less.

The foregoing description and examples has been set forth merely to illustrate the disclosure and are not intended as being limiting. Each of the disclosed aspects and embodiments of the present disclosure may be considered individually or in combination with other aspects, embodiments, and variations of the disclosure. In addition, unless otherwise specified, none of the steps of the methods of the present disclosure are confined to any particular order of performance. Modifications of the disclosed embodiments incorporating the spirit and substance of the disclosure may occur to persons skilled in the art and such modifications are within the scope of the present disclosure. Furthermore, all references cited herein are incorporated by reference in their entirety.

Terms of orientation used herein, such as “top,” “bottom,” “horizontal,” “vertical,” “longitudinal,” “lateral,” and “end” are used in the context of the illustrated embodiment. However, the present disclosure should not be limited to the illustrated orientation. Indeed, other orientations are possible and are within the scope of this disclosure. Terms relating to circular shapes as used herein, such as diameter or radius, should be understood not to require perfect circular structures, but rather should be applied to any suitable structure with a cross-sectional region that can be measured from side-to-side. Terms relating to shapes generally, such as “circular” or “cylindrical” or “semi-circular” or “semi-cylindrical” or any related or similar terms, are not required to conform strictly to the mathematical definitions of circles or cylinders or other structures, but can encompass structures that are reasonably close approximations.

Conditional language used herein, such as, among others, “can,” “might,” “may,” “e.g.,” and the like, unless specifically stated otherwise, or otherwise understood within the context as used, is generally intended to convey that some embodiments include, while other embodiments do not include, certain features, elements, and/or states. Thus, such conditional language is not generally intended to imply that features, elements, blocks, and/or states are in any way required for one or more embodiments or that one or more embodiments necessarily include logic for deciding, with or without author input or prompting, whether these features, elements and/or states are included or are to be performed in any particular embodiment.

Conjunctive language, such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

The terms “approximately,” “about,” and “substantially” as used herein represent an amount close to the stated amount that still performs a desired function or achieves a desired result. For example, in some embodiments, as the context may dictate, the terms “approximately”, “about”, and “substantially” may refer to an amount that is within less than or equal to 10% of the stated amount. The term “generally” as used herein represents a value, amount, or characteristic that predominantly includes or tends toward a particular value, amount, or characteristic. As an example, in certain embodiments, as the context may dictate, the term “generally parallel” can refer to something that departs from exactly parallel by less than or equal to 20 degrees.

Unless otherwise explicitly stated, articles such as “a” or “an” should generally be interpreted to include one or more described items. Accordingly, phrases such as “a device configured to” are intended to include one or more recited devices. Such one or more recited devices can be collectively configured to carry out the stated recitations. For example, “a processor configured to carry out recitations A, B, and C” can include a first processor configured to carry out recitation A working in conjunction with a second processor configured to carry out recitations B and C.

The terms “comprising,” “including,” “having,” and the like are synonymous and are used inclusively, in an open-ended fashion, and do not exclude additional elements, features, acts, operations, and so forth. Likewise, the terms “some,” “certain,” and the like are synonymous and are used in an open-ended fashion. Also, the term “or” is used in its inclusive sense (and not in its exclusive sense) so that when used, for example, to connect a list of elements, the term “or” means one, some, or all of the elements in the list.

Overall, the language of the claims is to be interpreted broadly based on the language employed in the claims. The language of the claims is not to be limited to the non-exclusive embodiments and examples that are illustrated and described in this disclosure, or that are discussed during the prosecution of the application.

Although systems and methods for whitening teeth have been disclosed in the context of certain embodiments and examples, this disclosure extends beyond the specifically disclosed embodiments to other alternative embodiments and/or uses of the embodiments and certain modifications and equivalents thereof. Various features and aspects of the disclosed embodiments can be combined with or substituted for one another in order to form varying modes of systems and methods for whitening teeth. The scope of this disclosure should not be limited by the particular disclosed embodiments described herein.

Certain features that are described in this disclosure in the context of separate implementations can be implemented in combination in a single implementation. Conversely, various features that are described in the context of a single implementation can be implemented in multiple implementations separately or in any suitable subcombination. Although features may be described herein as acting in certain combinations, one or more features from a claimed combination can, in some cases, be excised from the combination, and the combination may be claimed as any subcombination or variation of any subcombination.

While the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various embodiments described and the appended claims. Further, the disclosure herein of any particular feature, aspect, method, property, characteristic, quality, attribute, element, or the like in connection with an embodiment can be used in all other embodiments set forth herein. Any methods disclosed herein need not be performed in the order recited. Depending on the embodiment, one or more acts, events, or functions of any of the algorithms, methods, or processes described herein can be performed in a different sequence, can be added, merged, or left out altogether (e.g., not all described acts or events are necessary for the practice of the algorithm). In some embodiments, acts or events can be performed concurrently, e.g., through multi-threaded processing, interrupt processing, or multiple processors or processor cores or on other parallel architectures, rather than sequentially. Further, no element, feature, block, or step, or group of elements, features, blocks, or steps, are necessary or indispensable to each embodiment. Additionally, all possible combinations, subcombinations, and rearrangements of systems, methods, features, elements, modules, blocks, and so forth are within the scope of this disclosure. The use of sequential, or time-ordered language, such as “then,” “next,” “after,” “subsequently,” and the like, unless specifically stated otherwise, or otherwise understood within the context as used, is generally intended to facilitate the flow of the text and is not intended to limit the sequence of operations performed. Thus, some embodiments may be performed using the sequence of operations described herein, while other embodiments may be performed following a different sequence of operations.

Moreover, while operations may be depicted in the drawings or described in the specification in a particular order, such operations need not be performed in the particular order shown or in sequential order, and all operations need not be performed, to achieve the desirable results. Other operations that are not depicted or described can be incorporated in the example methods and processes. For example, one or more additional operations can be performed before, after, simultaneously, or between any of the described operations. Further, the operations may be rearranged or reordered in other implementations. Also, the separation of various system components in the implementations described herein should not be understood as requiring such separation in all implementations, and it should be understood that the described components and systems can generally be integrated together in a single product or packaged into multiple products. Additionally, other implementations are within the scope of this disclosure.

Some embodiments have been described in connection with the accompanying figures. Certain figures are drawn and/or shown to scale, but such scale should not be limiting, since dimensions and proportions other than what are shown are contemplated and are within the scope of the embodiments disclosed herein. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, any methods described herein may be practiced using any device suitable for performing the recited steps.

The methods disclosed herein may include certain actions taken by a practitioner; however, the methods can also include any third-party instruction of those actions, either expressly or by implication. For example, actions such as “positioning an electrode” include “instructing positioning of an electrode.”

In summary, various embodiments and examples of systems and methods for whitening teeth have been disclosed. Although the systems and methods for whitening teeth have been disclosed in the context of those embodiments and examples, this disclosure extends beyond the specifically disclosed embodiments to other alternative embodiments and/or other uses of the embodiments, as well as to certain modifications and equivalents thereof. This disclosure expressly contemplates that various features and aspects of the disclosed embodiments can be combined with, or substituted for, one another. Thus, the scope of this disclosure should not be limited by the particular disclosed embodiments described herein, but should be determined only by a fair reading of the claims that follow.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 1 V” includes “1 V.” Phrases preceded by a term such as “substantially” include the recited phrase and should be interpreted based on the circumstances (e.g., as much as reasonably possible under the circumstances). For example, “substantially perpendicular” includes “perpendicular.” Unless stated otherwise, all measurements are at standard conditions including temperature and pressure.

1. A method of cosmetically whitening teeth of a patient, the method comprising:
whitening the teeth at night using a carbamide peroxide-based whitening product, the carbamide peroxide-based whitening product having a concentration of carbamide peroxide of about 16% or less, wherein whitening the teeth at night comprises:
placing a quantity of the carbamide peroxide-based whitening product in a tray customized to the teeth of the patient;
placing the tray containing the quantity of the carbamide peroxide-based whitening product over the teeth of the patient before the patient sleeps; and
leaving the tray containing the quantity of the carbamide peroxide-based whitening product over the teeth of the patient for a length of time while the patient sleeps;
after at least one night of whitening the teeth at night using the carbamide peroxide-based whitening product, whitening the teeth during the day using a hydrogen peroxide-based whitening product, the hydrogen peroxide-based whitening product having a concentration of about 6% or less, wherein whitening the teeth during the day comprises:
placing a quantity of the hydrogen peroxide-based whitening product in the tray customized to the teeth of the patient;
placing the tray containing the quantity of the hydrogen peroxide-based whitening product over the teeth of the patient while the patient is awake during the day; and
leaving the tray containing the quantity of the hydrogen peroxide-based whitening product over the teeth of the patient for a length of time while the patient is awake during the day.

2. The method of cosmetically whitening teeth of a patient of claim 1, wherein the step of whitening the teeth at night using the carbamide peroxide-based whitening product is performed each night for two weeks.

3. The method of cosmetically whitening teeth of a patient of claim 2, wherein the step of whitening the teeth during the day using the hydrogen peroxide-based whitening product is performed after the step of whitening the teeth at night using the carbamide peroxide-based whitening product has been performed each night for two weeks.

4. The method of cosmetically whitening teeth of a patient of claim 3, wherein the step of whitening the teeth during the day using the hydrogen peroxide-based whitening product is performed each day for one week.

5. The method of cosmetically whitening teeth of a patient of claim 1, wherein the step of whitening the teeth during the day using the hydrogen peroxide-based whitening product is performed after the entire nighttime whitening protocol has been finished.

6. (canceled)

7. The method of cosmetically whitening teeth of a patient of claim 1, wherein the step of whitening the teeth at night using the carbamide peroxide-based whitening product is performed each night for between one and three weeks.

8. The method of cosmetically whitening teeth of a patient of claim 7, wherein the step of whitening the teeth during the day using the hydrogen peroxide-based whitening product is performed after the step of whitening the teeth at night using the carbamide peroxide-based whitening product has been performed each night for between one and three weeks.

9-22. (canceled)

23. The method of cosmetically whitening teeth of a patient of claim 1, wherein the carbamide peroxide-based whitening product comprises a chemical accelerant.

24. The method of cosmetically whitening teeth of a patient of claim 1, wherein the hydrogen peroxide-based whitening product comprises a chemical accelerant.

25. A method of cosmetically whitening teeth of a patient, the method comprising:
whitening the teeth at night using a nighttime whitening product, wherein whitening the teeth at night comprises:
placing a quantity of the nighttime whitening product in a tray customized to the teeth of the patient;
placing the tray containing the quantity of the nighttime whitening product over the teeth of the patient before the patient sleeps; and
leaving the tray containing the quantity of the nighttime whitening product over the teeth of the patient for a length of time while the patient sleeps;
after at least one night of whitening the teeth at night using the nighttime whitening product, whitening the teeth during the day using a daytime whitening product, wherein whitening the teeth during the day comprises:
placing a quantity of the daytime whitening product in the tray customized to the teeth of the patient;
placing the tray containing the quantity of the daytime whitening product over the teeth of the patient while the patient is awake during the day; and
leaving the tray containing the quantity of the daytime whitening product over the teeth of the patient for a length of time while the patient is awake during the day.

26. (canceled)

27. The method of cosmetically whitening teeth of a patient of claim 25, wherein the nighttime whitening product comprises a carbamide peroxide-based whitening product having a concentration of carbamide peroxide of between about 15%-22%.

28. The method of cosmetically whitening teeth of a patient of claim 25, wherein the daytime whitening product comprises a hydrogen peroxide-based whitening product having a concentration of hydrogen peroxide of between about 12%-18%.

29. The method of cosmetically whitening teeth of a patient of claim 25, wherein the daytime whitening product comprises a hydrogen peroxide-based whitening product having a concentration of hydrogen peroxide of about 12% and a carbamide peroxide-based whitening product having a concentration of carbamide peroxide of about 35%.

30. The method of cosmetically whitening teeth of a patient of claim 29, wherein the daytime whitening product comprises a ratio of about 75% of the hydrogen peroxide-based whitening product to about 25% of the carbamide peroxide-based whitening product.

31. The method of cosmetically whitening teeth of a patient of claim 25, wherein the step of whitening the teeth at night using the nighttime whitening product is performed each night for two weeks.

32. The method of cosmetically whitening teeth of a patient of claim 31, wherein the step of whitening the teeth during the day using the daytime whitening product is performed after the step of whitening the teeth at night using the nighttime whitening product has been performed each night for two weeks.

33-34. (canceled)

35. The method of cosmetically whitening teeth of a patient of claim 25, wherein the step of whitening the teeth during the day using the daytime whitening product is performed at least twice a day for at least 60 minutes.

36-51. (canceled)

52. The method of cosmetically whitening teeth of a patient of claim 25, wherein the nighttime whitening product comprises a chemical accelerant.

53. The method of cosmetically whitening teeth of a patient of claim 25, wherein the daytime whitening product comprises a chemical accelerant.

54. The method of cosmetically whitening teeth of a patient of claim 25, wherein the step of whitening the teeth during the day using the hydrogen peroxide-based whitening product is performed after the entire nighttime whitening protocol has been finished.
